SEQUENCE LISTING (3 sequences)

SEQ ID NO: 1 - DNA, 23 bp, Artificial Sequence
  A Fluorescein (FLC)-labeled ssDNA sequence (P1)
agtcagtgtggaaaatctctagc

SEQ ID NO: 2 - DNA, 23 bp, Artificial Sequence
  a synthetic oligonucleotide (T1)
gctagagattttccacactgact

SEQ ID NO: 3 - DNA, 23 bp, Artificial Sequence
  a single-base mismatch sequence (M1)
gctagagattgtccacactgact

(19) United States
(12) Patent Application Publication
(10) Pub. No.: US 2019/0144924 A1
(43) Pub. Date: May 16, 2019

(54) BIOSENSORS

(71) Applicant: Yissum Research Development Company of the Hebrew University of Jerusalem Ltd., Jerusalem (IL)

(72) Inventors: Meital RECHES, Bet Hasmonai (IL); Priyadip DAS, Jerusalem (IL)

(73) Assignee: Yissum Research Development Company of the Hebrew University of Jerusalem Ltd., Jerusalem (IL)

(21) Appl. No.: 15/775,186

(22) PCT Filed: Nov. 10, 2016
(86) PCT No.: PCT/IL2016/051212

§ 371 Date: May 10, 2018

Related U.S. Application Data
(60) Provisional application No. 62/253,977, filed on Nov. 11, 2015.

(51) Int. Cl. C12Q 1/6818

(52) U.S. Cl. CPC C12Q 1/6818

(57) ABSTRACT

Provided are molecular entities capable of transforming, aggregating or assembling into a three dimensional biosensors.

### TECHNOLOGICAL FIELD

The invention generally concerns methods and systems for use in the detection of target molecules such as nucleic acids.

### BACKGROUND

The detection of biomolecules such as DNA has attracted considerable research efforts mainly for detecting diseases and pathogens. For this purpose, numerous electrical, optical and microgravimetric sensors have been developed. Most of DNA detection methods are based on hybridisation and thus require the generation of single-stranded DNA prior to analysis. For example, to properly generate a signal, methods such as DNA microarrays, southern blotting, and in situ hybridization require denaturation of the double-stranded DNA into single strands, followed by their subsequent renaturation with specific probes. Following this strategy, a number of approaches have been developed in which the targeted DNA is detected directly. Since these approaches lack sequence specificity, developing simplified assays that can avoid such sequence specificity problems is required.

Electrochemistry-based detection methods may have great potential towards developing hand-held nucleic-acid analysis instruments. These devices demonstrate the implementation of in situ electrochemical (EC) detection method in a microfluidic flow-through EC-qPCR device. Their disadvantages reside in the fact that they are expensive, time consuming, and complicated to perform. The unique thermodynamics and specificity of molecular beacon-type probes in comparison with linear probes have led to their widespread use in protein-DNA interaction studies and in in vitro detection of RNA expression in living cells. The use of fluorescence techniques for developing assay systems for biomolecules is therefore the most convenient method due to their merits of high sensitivity, ease of use and their capability of in situ cellular imaging.

Among the several known nanoquenchers are carbon nanostructures including single-walled carbon nanotubes (SWNTs), graphene oxide (GO), and carbon nanoparticles (CNPs), which are water dispersible and can bind fluorescently labelled probes via hydrophobic and non-covalent π-π interactions. This binding results in emission quenching owing to fluorescence energy resonance transfer (FRET) and it has been successfully used to detect biomolecules. The preparation of some of these systems is often elaborate and it requires additional time and resources. Moreover, the toxicity safety for their use in clinical diagnostics is uncertain.

Metal complexes with their tunable coordination geometry and versatile redox and spectral properties provide a wide scope of possibilities in designing systems that are suitable for targeting DNA and proteins. Peptides with metal complexes on their side chains and peptide metal conjugates have been used to enhance or control the binding affinities of the peptides towards biomolecules. Several metal-peptide frameworks (MPFs) formed by different peptide families were reported. These MPFs have defined topologies that arise from the self-assembly properties of the peptides and their specific interactions with metals. In metal-peptide frameworks, specific metal-peptide interactions via metal-nitrogen and metal-oxygen binding, along with noncovalent interactions, are responsible for the formation of various nanostructures.

### GENERAL DESCRIPTION

Biomolecular detection has widespread applications in molecular diagnostics, industrial and environmental monitoring and civil defence. This has motivated the inventors to develop rapid, simple and cost-effective biosensors for a variety of target molecules, e.g., nucleic acids. The peptide-metal complexes and precursors thereof self-assemble into a variety of aggregate forms that may be tailored for use in a variety of detection methods. Detection methods utilizing the novel complexes of the invention are insensitive to contaminating materials, are fast, cost-effective and more reliable than many of the existing assays and detection methods.

Thus, in a first aspect, there is provided a molecular entity capable of transforming, aggregating or assembling into a three dimensional form that comprises a plurality of such molecular entities, each of said molecular entities being a biosensor comprising at least one peptide moiety and at least one moiety capable of altering fluorescence emission of a fluorescence beacon associated with the molecular entity. The altering of the emission, irrespective of its mechanism, may be to induce or quench such emission.

The invention further provides a biosensor comprising a peptide moiety and a moiety capable of altering fluorescence emission, the moiety comprising at least one metal ion, the biosensor being associated with at least one probe molecule having a fluorescent label, the probe molecule being selected to partially or fully interact with at least one target molecule.

In some embodiments, the peptide moiety and the moiety capable of altering fluorescence emission, comprising in some embodiments, as further detailed herein, a metal ion, are in a complex form.

The invention further provides a biosensor in a peptide-metal complex form, the complex comprising a peptide moiety; and a moiety capable of altering fluorescence emission, which comprises at least one metal ion, wherein the complex is associated with at least one labelled probe molecule selected to partially or fully interact with at least one target molecule.

In some embodiments, the complex is in a form selected from tubular structure; fibrilar structures; spheres; joint spherical structures; spherical aggregates; distorted spherical aggregates; linear, two-dimensional or three-dimensional arrays; and single molecule forms.

In some embodiments, the spheres are selected from microspheres and nanospheres and the tubular structures are selected from nanotubes and microtubes.

In some embodiments, the biosensor may be self-assembled into one or more three dimensional forms in solution; the forms being selected from tubular structure (tubes of various lengths and core diameter); fibrilar structures (fibrils); spheres (spherical arrangements); joint spherical structures (spherical arrangements associated to each other in a substantially linear form); spherical aggregates (aggregations of various sizes of spherical structures that are substantially spherical); distorted spherical aggregates (aggregations of various sizes of spherical structures that are substantially non-spherical); linear, two-dimensional or three-dimensional arrays (collections of single or mixed forms in an array form); and single molecule forms.

The self-assembled forms may be achievable by introducing the molecular biosensor, in a non-assembled form, to a solvent or solvent medium which induces self-assembly. Such solvents/solvent systems may be selected from protic polar solvents, which may be aqueous or non-aqueous. In some embodiments, the solvent/solvent system is selected from water and alcohols. In some embodiments, the solvent is selected from water, methanol, ethanol and mixtures thereof.

In some embodiments, the solvent is a solvent mixture comprising water and at least one alcohol.

In some embodiments, the solvent is methanol or ethanol or water/ethanol or water/methanol or water.

In some embodiments, the water/alcohol mixture comprises between 10 and 60% alcohol. In some embodiments, the water/alcohol mixture comprises between 10 and 50% alcohol. In some embodiments, the water/alcohol mixture comprises between 20 and 60% alcohol. In some embodiments, the water/alcohol mixture comprises between 10 and 50% alcohol.

The “peptide moiety” is an amino acid comprising moiety that is structured of one or more amino acids associated to each other in a linear fashion or a non-linear fashion, via a peptide bond, a modified peptide bond or a linker moiety. In some embodiments, the peptide moiety comprises one amino acid. In other embodiments, the peptide moiety comprises two or more amino acids, typically between 2 and 10 amino acids.

The amino acid(s) making up the peptide moiety may be any natural, synthetic or semi-synthetic amino acid. Such amino acids may be α- or β-forms, D- or L-amino acids or combinations thereof. The amino acid may be selected amongst alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine valine, pyrrolysine and selnocysteine; and amino acid analogs such as homo-amino acids, N-alkyl amino acids, dehydroamino acids, aromatic amino acids and α,α-disubstituted amino acids, e.g., cystine, 5-hydroxylysine, 4-hydroxyproline, a-aminoadipic acid, a-amino-n-butyric acid, 3,4-dihydroxyphenylalanine, homoserine, α-methylserine, ornithine, pipecolic acid, ortho, meta or para-aminobenzoic acid, citrulline, canavanine, norleucine, d-glutamic acid, aminobutyric acid, L-fluorenylalanine, L-3-benzothienylalanine and thyroxine.

In some embodiments, the amino acids are selected amongst aromatic amino acids, such as tryptophan, tyrosine, naphthylalanine, and phenylalanine. In some embodiments, the amino acid is phenylalanine or derivatives thereof.

In some embodiments, the phenylalanine derivatives is 4-methoxy-phenylalanine, 4-carbamimidoyl-1-phenylalanine, 4-chloro-phenylalanine, 3-cyano-phenylalanine, 4-bromo-phenylalanine, 4-cyano-phenylalanine, 4-hydroxymethyl-phenylalanine, 4-methyl-phenylalanine, 1-naphthyl-alanine, 3-(9-anthryl)-alanine, 3-methylphenyl alanine, m-amidinophenyl-3-alanine, phenylserine, benzylcysteine, 4,4-biphenylalanine, 2-cyano-phenylalanine, 2,4-dichloro-phenylalanine, 3,4-dichloro-phenylalanine, 2-chloropenylalanine, 3,4-dihydroxy-phenylalanine, 3,5-dibromo tyrosine, 3,3-diphenyl alanine, 3-ethyl-phenylalanine, 3,4-difluoro-phenyl alanine, 3-chloro-phenylalanine, 3-chloro-phenylalanine, 2-fluoro-phenylalanine, 3-fluorophenyl alanine, 4-amino-L-phenylalanine, homophenylalanine, 3-(8-hydroxyquinolin-3-yl)-1-alanine, 3-iodo-tyrosine, kynurenine, 3,4-dimethyl-phenylalanine, 2-methyl-phenylalanine, m-tyrosine, 2-naphthyl-alanine, 5-hydroxy-1-naphthalene, 6-hydroxy-2-naphthalene, meta-nitro-tyrosine, (beta)-beta-hydroxy-1-tyrosine, (beta)-3-chloro-beta-hydroxy-1-tyrosine, o-tyrosine, 4-benzoyl-phenylalanine, 3-(2-pyridyl)-alanine, 3-(3-pyridyl)-alanine, 3-(4-pyridyl)-alanine, 3-(2-quinolyl)-alanine, 3-(3-quinolyl)-alanine, 3-(4-quinolyl)-alanine, 3-(5-quinolyl)-alanine, 3-(6-quinolyl)-alanine, 3-(2-quinoxalyl)-alanine, styrylalanine, pentafluoro-phenylalanine, 4-fluoro-phenylalanine, phenylalanine, 4-iodo-phenylalanine, 4-nitro-phenylalanine, phosphotyrosine, 4-tert-butylphenylalanine, 2-(trifluoromethyl)-phenylalanine, 3-(trifluoromethyl)phenyl alanine, 4-(trifluoromethyl)-phenylalanine, 3-amino-L-tyrosine, 3,5-diiodotyrosine, 3-amino-6-hydroxy-tyrosine, tyrosine, 3,5-difluoro-phenylalanine and/or 3-fluorotyrosine.

In some embodiments, the peptide moiety comprises at least two amino acids, at least one of which being an aromatic amino acid.

As stated above, the amino acids in the peptide moiety may be associated to each other via a peptide bond, as known in the art, a modified bond that may or may not be derived from a peptide bond or via a linker moiety. For certain applications, the peptide bond may be replaced or modified such that it is a non-naturally occurring bond obtained, e.g., by reduction (to —CH₂—NH—), alkylation of the nitrogen atom (e.g., by one or more carbon containing group), replaced by amidic bond, urea bonds, sulfonamide bond, etheric bond (—CH₂—O—), thioetheric bond (—CH₂—S—), or by —CS—NH.

In some embodiments, the amino acids making up the peptide moiety are linked to each other via a linker moiety selected amongst carbon-containing groups. Such linker moieties may be aliphatic or contain one or more double or triple bonds. In some embodiments, the linker is a C₁-C₅ alkylene, such as methylene, ethylene, propylene, butylene and pentylene. In other embodiments, the linker is of the general structure:

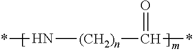

wherein
each * denotes a point of connectivity to an amino acid group;
n is between 0 and 4; and
m is between 1 and 4.

In some embodiments, the peptide moiety comprises an amino acid selected from phenylalanine (Phe) and glycine (Gly). In some embodiments, the peptide moiety comprises two or more amino acids, each amino acid being connected, bonded, or associated to at least one other amino acid via a peptide bond or a linker moiety having one or more carbon groups. In some embodiments, the linker moiety is a methylene or ethylene moiety. In some embodiments, the peptide moiety is a compound herein designated Compound 6.

In some embodiments, the peptide moiety comprises the amino acid motif Phe-Gly or Gly-Phe.

The “moiety capable of altering fluorescence emission” is a moiety present on or associated with, or bonded to the peptide moiety and which influences fluorescence emission from a fluorescent group present or associated with the complex or the peptide. Such a moiety may alter fluorescence emission through an energy transfer mechanism, that may involve attenuation, quenching (complete or partial), enhancement, shift in wavelength, shift in polarity, change in fluorescence lifetime and other mechanisms. One example of a fluorescence-modifying group is a quenching group. In some embodiments, the moiety is selected to quench fluorescence associated with a fluorescent group or a tag or a label. The “fluorescence-quenching moiety” is thus a functional group that attenuates fluorescence emission, leading to an emission signal that is less intense than expected or which is completely absent. Quenching occurs through an energy transfer between the fluorescent group and the quenching group.

In some embodiments, the molecular entities in a biosensor of the invention comprise a single moiety capable of altering fluorescence emission. In some embodiments, the molecular entities comprise each two or more moieties capable of altering fluorescence emission, at least one of which being positioned at one of the peptide terminus.

The fluorescence quenching moiety may be selected from aliphatic amines, aromatic amines, halogenated moieties, electron scavengers and others. In some embodiments, the fluorescence quenching moiety is selected from aromatic amines and electron scavengers. In some embodiments, the aromatic amines are selected from pyridine-based moieties, indole-based moieties, pyridinium-based moieties, imidazolinium-based moieties, purine-based moieties, pyrimidine and triazine moieties may be used. In some embodiments, the electron scavengers are moieties comprising at least one metal ion, the metal being selected from Cu, Cd, Pd, Mn, Eu, As, Cs, Zn, Hg, Ni and Co.

In some embodiments, the metal is Cu or Cd or Pd or Mn or Eu or As or Cs or Zn or Hg or Ni or Co. In some embodiments, the metal is Cu or Cd or Pd or Mn or Eu or As or Cs or Zn. In some embodiments, the metal is Cu or Cd or Pd or Mn or Eu or As or Cs. In some embodiments, the metal is Cu or Cd or Pd or Mn or Eu or As. In some embodiments, the metal is Cu or Cd or Pd or Mn or Eu. In some embodiments, the metal is Cu or Cd or Pd or Mn. In some embodiments, the metal is Cu or Cd or Pd. In some embodiments, the metal is Cu or Cd. In some embodiments, the metal is Cu.

In some embodiments, the metal ion being part of the moiety capable of altering fluorescence emission is an electron scavenger and thus acts as a fluorescence quenching moiety.

In some embodiments, the at least one metal ion may be directly associated with an amino acid of the peptide moiety or with a ligand group associated with an amino acid of the peptide moiety. In some embodiments, the moiety capable of altering fluorescence emission is a metal ion associated with at least one ligand moiety; the ligand moiety being a metal coordination group, optionally comprising at least one aromatic amine moiety.

In some embodiments, the metal coordination group is a ligand moiety comprising at least one aromatic or heteroaromatic (heteroaryl) group. In some embodiments, the heteroaromatic group comprises one or more nitrogen atoms. In some embodiments, the heteroaryl group is pyridine. In some embodiments, the ligand moiety is 2,2′-dipicolylamine or any derivative thereof.

As used herein, the peptide-metal complex refers to a biosensor according to the invention wherein the peptide moiety and the moiety capable of altering fluorescence emission, that are associated or bonded to one another via a covalent bond, are associated to or characterized by the presence of a ligand moiety or a metal coordination moiety which holds in a form of a metal complex, at least one metal ion. Biosensors of the invention are not all peptide-metal complexes and may comprise one or more moieties capable of altering fluorescence emission that are not or do not comprise a metal ion.

Where a metal ion is present, in some embodiments, the ligand moiety is selected to coordinate a metal ion selected from Cu, Cd, Pd, Mn, Eu, As, Cs Hg, Ni, Co and Zn. In some embodiments, the metal ion is selected from Cu, Cd, Pd, Mn, Eu, As and Cs. In some embodiments, the metal ion is selected from Cu, Cd, Pd, Mn, Eu and As. In some embodiments, the metal ion is selected from Cu, Cd, Pd, Mn and Eu. In some embodiments, the metal ion is selected from Cu, Cd, Pd and Mn. In some embodiments, the metal ion is selected from Cu, Cd and Pd. In some embodiments, the metal ion is selected from Cu and Cd. In some embodiments, the metal ion is Cu.

In some embodiments, the metal ion is selected from Cu, Cd, Pd, Mn, Eu, As and Cs and the ligand moiety comprises at least one nitrogen containing heteroaromatic ring structure. In some embodiments, the heteroaryl group is pyridine. In some embodiments, the ligand moiety is 2,2′-dipicolylamine or any derivative thereof.

In some embodiments, the biosensor is based or comprises a compound herein designated Compound L and Compound LM, wherein the metal ion depicted in Compound LM may be Cu, as depicted hereinbelow or in the figures, for the sake of simplicity, or any metal ion, or any metal ion selected from Cu, Cd, Pd, Mn, Eu, As, Cs Hg, Ni, Co and Zn.

Thus, the invention further provides a peptide-metal complex comprising a peptide moiety comprising at least one amino acid; and a moiety capable of altering fluorescence emission, e.g., a fluorescence-quenching moiety, comprising at least one metal ion, said moiety being associated with said peptide moiety.

A peptide-metal complex is structured of a peptide moiety comprising at least one amino acid and a ligand group capable of associating with the at least one metal ion. In some embodiments, the peptide-metal complex is designed to have a structure of the Formula I: A′-B-A, wherein A and A′ are each, independently of the other, a moiety capable of altering fluorescence emission, e.g., a fluorescence-quenching moiety, such that at least one of A and A′ may be absent; and B is a peptide moiety comprising at least one amino acid.

In some embodiments, the peptide-metal complex of the general Formula I, wherein A and A′ are each, independently of the other, a moiety capable of altering fluorescence emission, e.g., a fluorescence-quenching moiety, comprising a coordination moiety and at least one metal ion, such that at least one of A and A′ may be absent; and B is a peptide moiety comprising at least one aromatic amino acid. As detailed herein, the complex is further associated with a labelled probe molecule that is selected to be capable of association, partially or fully, with at least one target molecule, as defined.

The biosensors of the invention self-assemble into a variety of material forms (assemblies) by means of spontaneous or solvent-driven transformations. As further exemplified and detailed below, these assemblies, as with the single molecular forms, are suitable for (optical) detection of target molecules such as nucleic acids. The assemblies are selected from spheres, such as nanospheres and microspheres; neck structures, such as joint spherical structures; spherical aggregates; distorted spherical aggregates; linear, two-dimensional or three-dimensional arrays; tubular structures, fibrilar structures and other aggregated forms.

Thus, the invention further provides a self-assembly of a biosensor, e.g., in the form of a peptide-metal complex according to the invention. In some embodiments, the self-assembly is selected as above.

The self-assembled forms as well as the single molecular peptide-metal complexes of the invention are suitable for use as biosensors, namely as detection or diagnostic tools in the optical detection of nucleic acid molecules.

In another aspect of the invention, there is provided a spherical biosensor, the biosensor being comprised of a plurality of peptide-metal complexes according to the invention.

In some embodiments, the biosensor is a complex according to Formula I.

In some embodiments, the biosensor is a self-assembled form of a biosensor of Formula I, as detailed herein. In some embodiments, the self-assembled from is a sphere or a spherical aggregation of spheres. In some embodiments, the sphere is a nanosphere.

The biosensors of the invention may be utilized in a variety of optical method for detecting the presence of a target molecule, e.g., a nucleic acid, in a sample. The nucleic acid to be detected may be a natural, artificial or analog of nucleic acids, or combinations thereof, including DNA, such as single stranded DNA (ssDNA), double-stranded DNA (dsDNA), cDNA; and RNA. The detection of a target nucleic acid by a biosensor according to the invention is enabled, as further explained hereinbelow, by associating a biosensor, e.g., a peptide-metal complex or a self-assembled form thereof, according to the invention, to at least one labelled (tag) probe molecule that is selected to coordinate, associate, aggregate, hybridize or generally bond to the target nucleic acid which detection in a sample is desired.

The probe molecule may be any compound or moiety that is associated with a tag or a label that is optically-detectable, which produces, or causes to produce, fluorescence, luminescence, light or color that is detectable. In some embodiments, the detectable label is selected to produce fluorescence or light or a specific wavelength as may be desired by the practitioner.

The probe molecule may be a nucleic acid or any other moiety or material or agent that is capable of selective binding to a target nucleic acid (such as PNA, morpholinos, and others). In some embodiments, the probe molecule is a nucleic acid selected to hybridize to the target nucleic acid through a portion or portions of the probe sequence that are substantially complementary to a target nucleic acid sequence.

Thus, the invention further provides a peptide-metal complex or self-assembled from thereof associated with at least one probe molecule, being in some embodiments, a nucleic acid, optionally labelled with a tag or an optically active moiety or group.

In some embodiments, the probe nucleic acid and/or the target nucleic acid is selected from aptamers, ribonucleotides, deoxyribonucleotides, ribonucleotide polyphosphate molecules, deoxyribonucleotide polyphosphate molecules, peptide nucleotides, nucleoside polyphosphate molecules, metallonucleosides, phosphonate nucleosides and modified phosphate-sugar backbone nucleotides. In some embodiments, the probe nucleic acid is selected to hybridize to the target nucleic acid that is selected from ribonucleotides, deoxyribonucleotides, ribonucleotide polyphosphate molecules, deoxyribonucleotide polyphosphate molecules, peptide nucleotides, nucleoside polyphosphate molecules, metallonucleosides, phosphonate nucleosides and modified phosphate-sugar backbone nucleotides.

The fluorescent-labelled or labelled probe nucleic acid, when associated with the complex or self-assembled form exhibits a fluorescence emission that is quenched. It is only upon hybridization of the probe nucleic acid to the target nucleic acid, that the probe nucleic acid undergoes a conformational change, causing ejection of the fluorescent tag and dequenching of fluorescence.

Non-limiting examples of optical tag moieties that may be associated with a probe molecule, e.g., probe nucleic acid, are: YO-PRn™-1, YDYO™-1, Calrein, FITC, FluorX™, Alexa™, Alexa™ 546, Alexa™ 594, Rhodamine 110, Rhodamine Green™, Rhodamine 123, Rhodamine Phalloidin, Rhodamine B, Rhodamine Red™, Oregon Green™ 500, Oregon Green™ 488, RiboGreen™, Magnesium Green™, Magnesium Orange™, Calcium Green™, Calcium Orange™, Calcium Crimson™, TO-PRO™-1, TO-PRO™-3, JOE, BODIPY530/550, Dil, BODIPY TMR, BODIPY558/568, BODIPY564/570, TRITC, Phycoerythrin R, Phycoerythrin B, Pyronin Y, TAMRA, ROX, Texas Red, Nile Red, YO-PRO™-3, YOYO™-3, R-phycocyanin, C-Phycocyanin, TOT03, DiD DilC(5), Cy3™, Cy3.5™, Cy5™, Cy5.5, Thiadicarbocyanine, HEX, TET, CAL Fluor Gold 540, CAL Fluor Orange 560, CAL Fluor Red 590, CAL Fluor Red 610, CAL Fluor Red 635, FAM, Fluorescein, Fluorescein-C3, Pulsar 650, Quasar 570, Quasar 670 and Quasar 705.

In some embodiments, the tag or label moiety is a fluorescein-based tag.

Suitable pairs of tag-quencher are known in the art, for example in: White et al., Fluorescence Analysis: A Practical Approach (Marcel Dekker, New York, 1970); Handbook of Fluorescence Spectra of Aromatic Molecules, 2nd Edition (Academic Press, New York, 1971); Griffiths, Color and Constitution of Organic Molecules (Academic Press, New York, 1976); Haugland, Handbook of Fluorescent Probes and Research Chemicals (Molecular Probes, Eugene, 1992); Pringsheim, Fluorescence and Phosphorescence (Interscience Publishers, New York, 1949); Haugland, R. P., Handbook of Fluorescent Probes and Research Chemicals, 6th Edition (Molecular Probes, 1996) and others.

In some embodiments, the probe nucleic acid is ssDNA and the target nucleic acid is selected from ssDNA and dsDNA.

In some embodiments, the probe nucleic acid is dsDNA and the target nucleic acid is a ssDNA.

Hybridization of the probe nucleic acid to the target nucleic acid yields a double-stranded nucleic acid from complementary single stranded nucleic acids or a triplex nucleic acid. The hybridization may occur between two nucleic acid strands perfectly matched or substantially matched with any number of mismatches.

The biosensor of the invention may be prepared by a method comprising:
a) obtaining a biosensor, e.g., a peptide-metal complex or self-assembled form thereof;
b) associating said biosensor or self-assembled form thereof with at least one probe molecule, optionally associated with at least one tag or label.

In some embodiments, the peptide-metal complex is formed by reacting a peptide moiety with at least one moiety capable of altering fluorescence emission, as defined herein. Optionally, the self-assembled complex is formed by treating the peptide-metal complex under conditions permitting conversion, aggregation or assembly of a molecular complex into a self-assembled complex as defined herein. FIG. 4 depicts a detection method of a nucleic acid in a sample, utilizing a biosensor according to the invention.

In FIG. 4, a biosensor in the form of a molecular entity

is self-assembled into a spherical shape biosensor that comprises a plurality of molecular entities. The spherical biosensor is designated
and is structured of a plurality of said molecular entities. Once formed, the biosensor may be utilized in a variety of detection methods and for a variety of purposes. As mentioned above, the detection of a target nucleic acid by a biosensor is enabled by associating the molecular entity, e.g., peptide-metal complex, or a self-assembled form thereof, e.g., the spherical biosensor, to at least one labelled (tag) probe molecule that is selected to coordinate, associate, aggregate, hybridize or generally bond to the target nucleic acid which detection in a sample is desired. The labelled probe molecule,
being in some embodiments, a DNA labelled with at least one moiety capable of altering fluorescence emission, e.g., a fluorescence-quenching moiety, exhibits fluorescence emission when not associated to said biosensor. Upon contacting of a sample suspected of comprising a target nucleic acid with a biosensor of the invention, if a target nucleic acid is indeed present in the sample, hybridization or otherwise association of the probe molecule to the target nucleic acid will cause release of the labelled probe molecule from the biosensor and subsequent dequenching of the fluorescence emission. The emission indicates association of the probe molecules to the target nucleic acids, thereby indicating presence of the target nucleic acids in the sample.

The emission may be detected and quantified according to methods known in the art.

The contacting of a sample with a biosensor of the invention may take place in solution, namely when one or both of the biosensor and target nucleic acid are in solution, or when the biosensor is immobilized on a matrix or a substrate.

As methods of the invention utilizing a complex or a self-assembled form are not limited to purified or low-contaminants samples, the methods may be used on a wide spectrum of biological samples such as blood, buccal/saliva, menstrual blood, skin, semen and vaginal fluids. The methods may be similarly applied to foods and beverages and samples derived from plants and marine materials.

Methods of the invention are thus not limited to any one specific field and may be utilized as assays in forensic science, in medicine, in agriculture science, in food science, in the general diagnostic arena, and may be tailored, by suitable selectins of biosensors, e.g., peptides and quenching moieties, as well as probe molecules and labels, to any other diagnostic use.

In some embodiments, the methods are used in the detection of various disease states.

The invention further provides a diagnostic kit, the kit comprising a biosensor according to the invention and instructions of use. In some embodiments, the kit comprises in two or more separate containers a peptide-metal complex in a free molecular form or in a self-assembled form and a labelled or unlabelled probe molecule and at least one agent and instructions for enabling association between the free molecular form or self-assembled form and the labelled or unlabelled probe molecule.

The invention further provides a compound herein designated Compound 6, Compound L and Compound LM.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 depicts an exemplary synthetic methodology adopted for the synthesis of biosensors of the invention referred to herein as L and LM.

FIGS. 2A-R provide microscopic analysis of the self-assembled structures formed by L and LM. Left-hand panels: representative TEM micrographs; middle panels: HR-SEM micrographs; and right-hand panel: AFM micrographs (two-dimensional representation) of the self-assembled structures formed by L and LM under different solvents (see for example Table 1 for details): FIGS. 2A, B, C—condition 1, FIGS. 2D, E, F—condition 2, FIGS. 2G, H, I—condition 3, FIGS. 2J, K, L—condition 4, FIGS. 2M, N, O—condition 5 and FIGS. 2P, Q, R—condition 6.

FIGS. 3A-E depicts self-assembly of LM in different solvents. FIG. 3A—schematic representation of the various self-assembled structures formed by LM in different solvents. FIG. 3B—UV-vis absorbance spectra of LM (3 mg/mL) in methanol (line 1), 50% ethanol (line 2), and water (line 3). Deconvoluted FT-IR spectra of self-assembled LM in (FIG. 3c) methanol, (FIG. 3d) 50% ethanol and (FIG. 3e) water. The dashed line indicates the original FTIR spectra and the solid line represents the deconvoluted curves with a Gaussian function.

FIG. 4 depicts by way of an illustration DNA detection by LM. This is a schematic representation of an embodiment of the invention directed at detecting target HIV DNA (T1) using LM as a sensing platform. In the first step, the fluorescein-labelled probe DNA (P1) interacts and adsorbs on the self-assembled aggregated spherical structures of LM; this triggers the florescence quenching. Then, the presence of the target DNA (T1) induces fluorescence recovery owing to the conformational change of P1 (dye-labelled SS DNA) into a duplex form.

FIGS. 5A-D provide (FIG. 5A) fluorescence spectra of the probe DNA P1 (50 nM) under different conditions (excitation at 494 nm): (1) P1 in tris-HCl buffer (pH =7.4); (2) P1+400 nM HIV DNA (T1); (3) P1+0.016 mM LM+400 nM HIV DNA (T1); (4) Probe DNA+0.016 mM LM. (FIG. 5B) Fluorescence intensity ratio of P1 (red markers) and P1-LM (blue markers) with (F/F₀-1) plotted against the logarithm of the concentration (Log C) of HIV1 DNA (T1). Excitation: 494 nm, emission: 521 nm. (FIG. 5C) The effect of LM dosage (0.539 mM) on the emission intensity of the probe DNA (P1) (50 nmol L⁻¹). (FIG. 5D) Fluorescence quenching of P1 (50 nm) in Tris-HCl buffer by LM as a function of time (green markers), and fluorescence restoration of P1-LM in Tris-HCl buffer (pH=7.4) by T1 (HIV1) (200 nM) as a function of time (grey markers). Excitation: 494 nm, and emission: 521 nm. SD was calculated based on 3 experiments.

FIGS. 6A-D depict optimisation of the detection system. FIG. 6A—Fluorescence spectra of the fluorescein-labelled probe DNA-LM conjugate (P1-LM) after incubation with increasing concentrations of target HIV DNA (T1) ranging from 0 to 500 nM, at room temperature; excitation wavelength=494 nm; incubation time=180 min. FIG. 6B—The graph plots (F_{T}-F_{M})/F_{M} (emission recovery) versus target-DNA (T1) concentrations. [probe DNA]=50 nmol, [LM]=0.016 mM, incubation time=180 min; emission intensities are measured at 521 nm. FIG. 6C—Fluorescence intensity changes (F_{T}/F_{M}-1) of the fluorescein-labelled probe DNA (P1)+LM towards the target DNA (T1A, 100 nM), (T1B, 200 nM) and single-base mismatched DNA (MT1, 1 μM). F_{M} and F_{T} are the emission intensities of P1-LM at 521 nm in the absence and presence of different targets. FIG. 6D—Fluorescence response (F/F₀) of P1-LM incubated in the presence of 500 nM T1 with or without (control) several interfering proteins at a concentration of 2 μM; F and F₀ are the emission intensities of P1-LM/T1 and P1-LM at 521 nm, respectively.

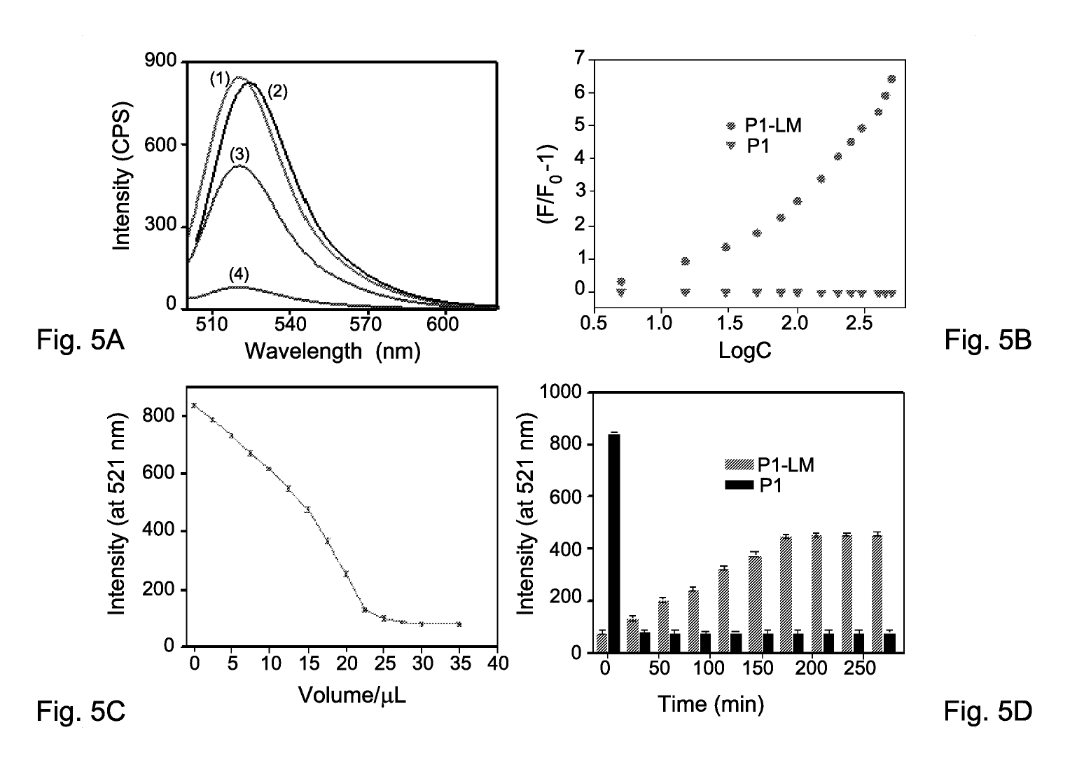

FIGS. 7A-B depict size distribution obtained from the DLS measurement for the spherical particles formed by (FIG. 7A) LM in methanol and (FIG. 7B) L in 50% ethanol.

FIGS. 8A-C show (FIG. 8A) TEM micrograph of self-assembled structures formed by LM in 50% ethanol. HR-SEM micrographs of (FIG. 8B) self-assembled structures formed by LM in water and (FIG. 8C) L in water.

### DETAILED DESCRIPTION OF EMBODIMENTS

### Results and Discussion

### Synthesis and Characterization

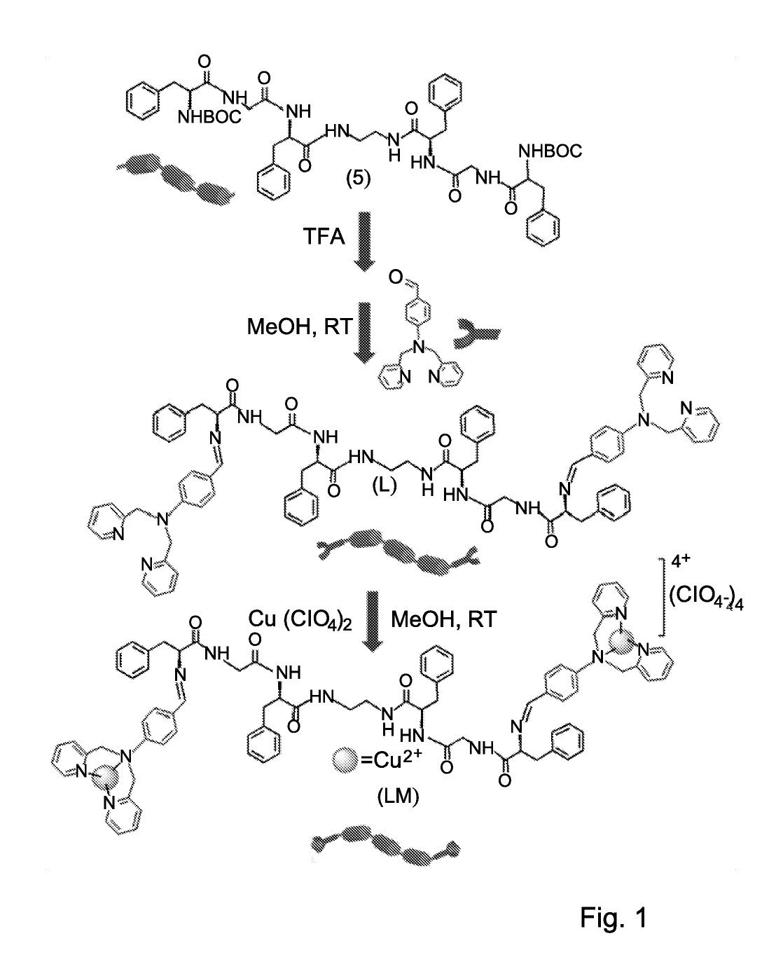

As depicted in FIG. 1, for exemplification purposes, to generate a complex of the invention, molecule (5) that comprises two repeating units of Phe-Gly-Phe (Phe=phenylalanine and Gly=glycine) linked by an ethylene diamine linker (see Experimental below). This exemplary peptide unit was chosen because it was assumed that the aromatic Phe will interact with DNA fragments through aromatic interactions. In addition, the free NH group will promote intermolecular hydrogen bonding and together with aromatic interaction, it will lead to the peptide's self-assembly. It was further assumed that the ethylene diamine bridge will act as a flexible linker of the two peptides, which will provide structural freedom in the self-assembly process. The de-protected N-terminus of 5 was then coupled to the metal chelator 4-(bis(pyridin-2-ylmethyl)amino)-benzaldehyde to produce the ligand (L). Considering the metal binding affinity of the 2,2′-dipicolylamine (DPA)-based receptor, the metal-peptide conjugate (LM, ligand-metal conjugate) was synthesized by reacting an aqueous solution of Cu(ClO₄)₂ with a methanol solution of L. All synthesized products were isolated and characterised by standard analytical techniques as well as spectroscopic and biophysical methods.

In addition, all UV-Vis absorption spectra of L and LM were recorded in order to detect the change in the optical spectral pattern of the peptide (L) in the presence and absence of the metal ion.

To trigger the self-assembly of L and LM, the two compounds were dissolved in different solvents having different polarities. First, the compound (either L or LM) was dissolved in 1,1,1,3,3,3-hexafluoro-2-propanol (HFP) to a concentration of 100 mg/mL. Then, each solution was diluted with methanol, 50% ethanol or water (Table 1). The polarized solvents (methanol<<50% ethanol<<water) allowed the peptides to self-assemble.

TABLE 1
The different self-assembly conditions for L and LM
and the resulting assemblies

| Condition | Compound | Solvent | Concentration | Assemblies |
|---|---|---|---|---|
| 1 | L | Methanol | 2 or 3 mg/mL | No distinct |
| | | | | structures |
| 2 | L | 50% | 2 or 3 mg/mL | Spheres |
| | | Ethanol | | |
| 3 | L | Water | 2 or 3 mg/mL | Distorted |
| | | | | spherical |
| | | | | aggregates |
| 4 | LM | Methanol | 2 or 3 mg/mL | Spheres |
| 5 | LM | 50% | 2 or 3 mg/mL | Joint |
| | | Ethanol | | spherical |
| | | | | structures |
| 6 | LM | Water | 2 or 3 mg/mL | Spherical |
| | | | | aggregates |

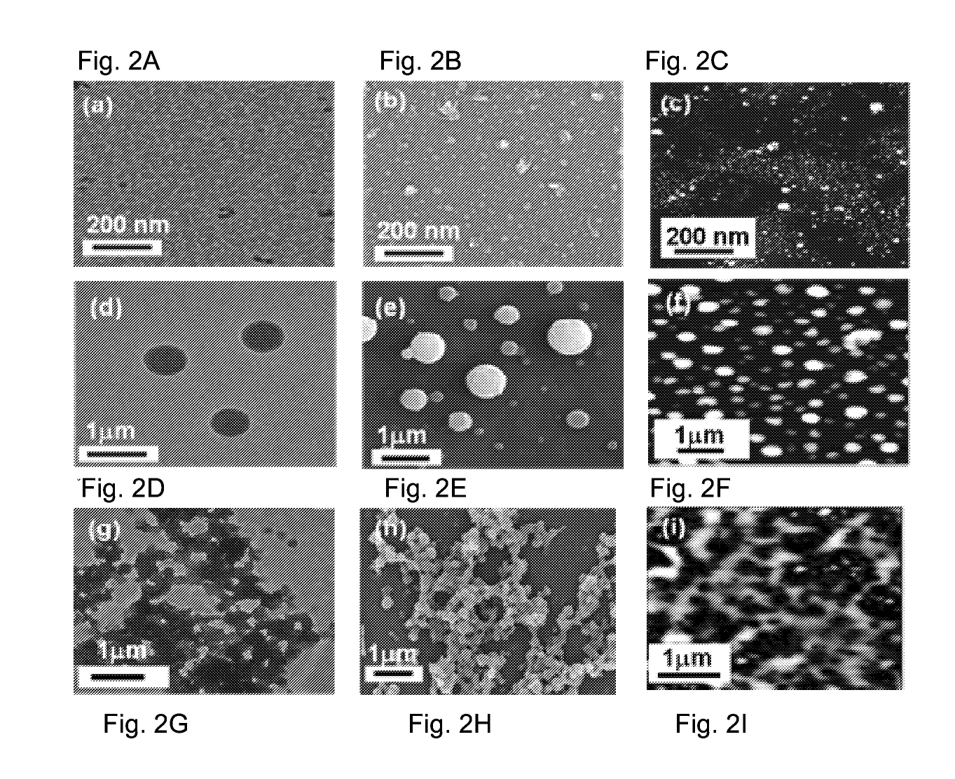

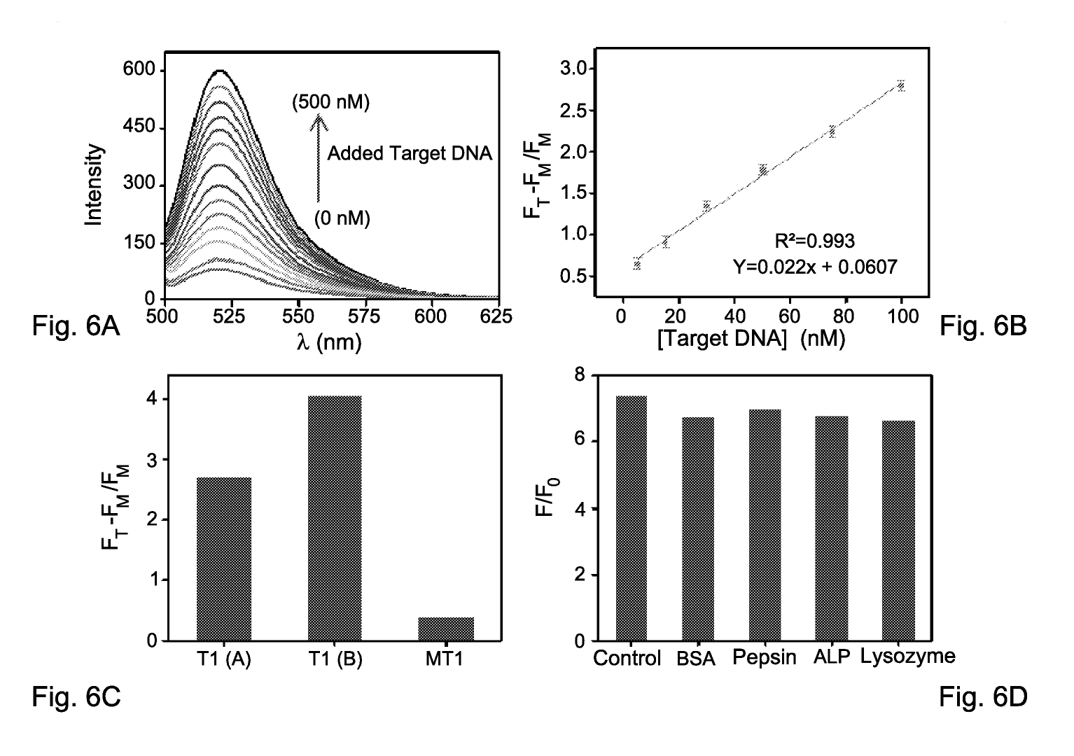

High-resolution scanning electron microscopy (HR-SEM) analysis revealed that in methanol L did not form any distinct structures (FIG. 2B) but LM can form spherical assemblies with nanomeric dimensions (FIG. 2K). Dynamic light scattering (DLS) analysis revealed that the average diameter of the particles was 714±27 nm (FIG. 7A). In 50% ethanol, L self-assembled into spherical nanostructures (average diameter 526±13 nm; FIG. 7B and FIG. 2E), whereas LM formed nanospheres that were joined together (FIG. 2N). It had been suggested that for such a structure, the nanospheres are first arranged in an ordered array and then commutate with each other through neck formation.

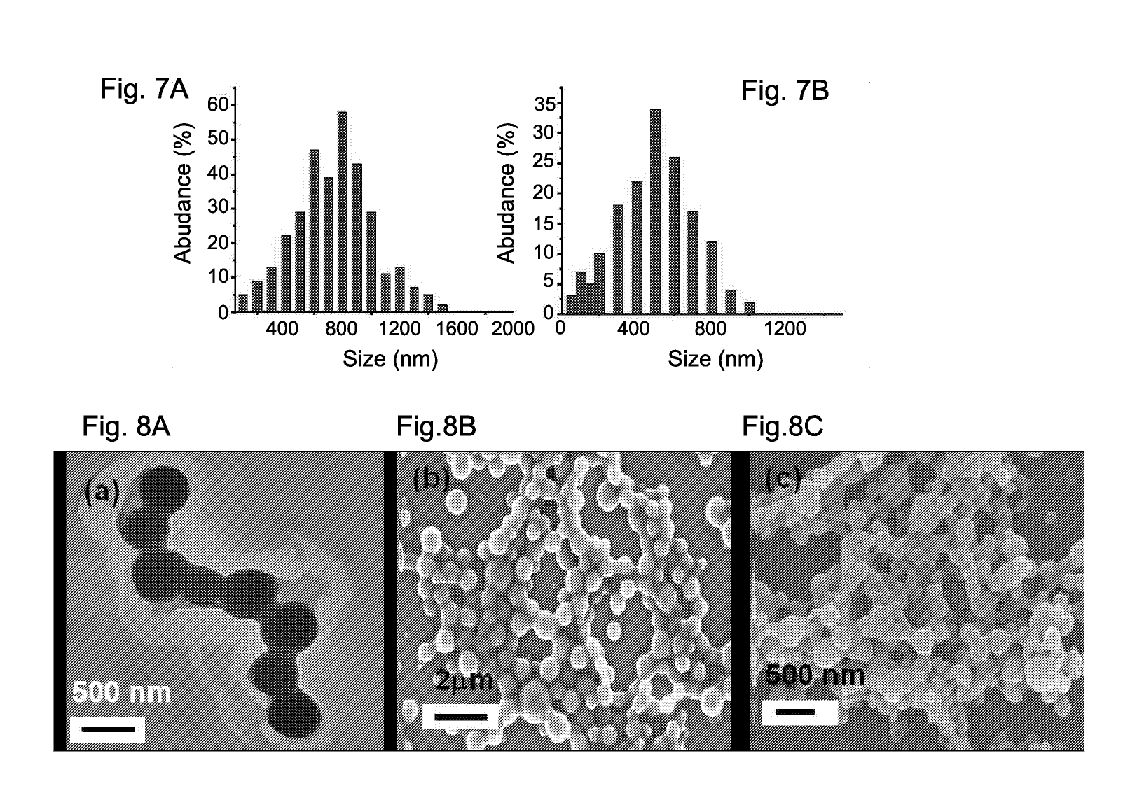

For further insights into the morphology of the self-assembled nanomeric spherical structures formed by L and LM under different conditions of solvents, transmission electron microscopy (TEM) analysis was performed. This analysis revealed a difference between the nanospheres formed under conditions 2 and 4; Table 1 (FIG. 2D and J): The spheres formed under condition 4; Table 1 by LM had a comparable lighter contrast with a white center and look like a hollow sphere, which suggests a lower electron density. The TEM analysis of the self-assembled structures formed under condition 5; Table 1, further confirmed the morphology of joined nanospheres through neck formation (FIG. 2M, FIG. 8A).

AFM analysis further supported the results obtained by HR-SEM and TEM as different nanostructures formed by L and LM in different solvents. Under condition 2 (Table 1), the nanospheres formed by L in 50% ethanol had a height ranging from 5 nm to 32 nm. (FIG. 2F, FIG. 9A-C). The height of the spheres formed by LM in 100% methanol (condition 4, Table 1) ranged from 11 nm to 79 nm (FIG. 2, FIGS. 9D-F). These findings are in good agreement with the results obtained by the DLS measurements. Under condition 5, where the nanospheres communicated with each other through neck formation, the height of the spheres varied from 92 nm to 434 nm (FIG. 2O, FIGS. 9G-I). From the height analysis, it is clear that the spheres formed by LM in 50% ethanol (condition 5) are much higher than the spheres formed by LM under condition 4 and by L under condition 2. When the self-assembly of L and LM was examined in water, it was found that both L and LM self-assembled into a 3D network of aggregated spheres. The spherical structures in the network formed by L and LM were different in size and morphology. L formed 3D aggregates of distorted spherical assemblies (FIGS. 2G-I) when compared with LM (FIG. 2P-R).

Overall, both L and LM formed spherical structures with diverse morphologies in solvents having different polarities. The self-assembly process is controlled by the molecular parameters of the peptide-based monomer, which depends on peptide-peptide and metal-peptide interaction energies that govern the stabilities and average size of the self-assembled structures.

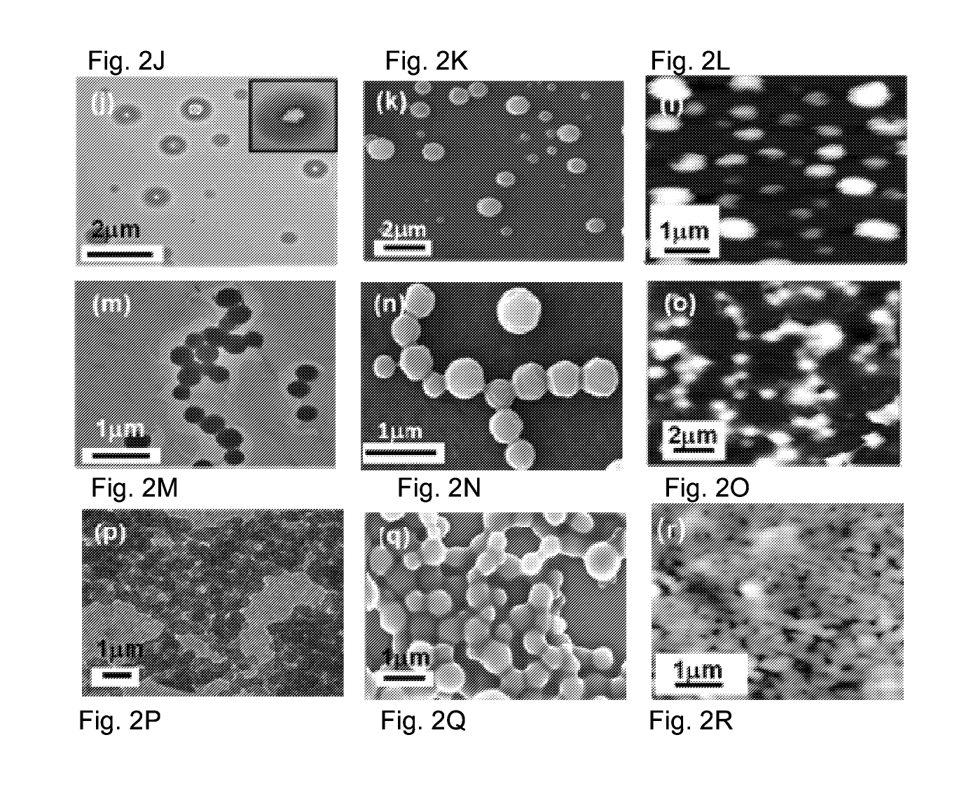

The structural transformation of peptide assemblies is dependent on the solvent's polarity. This may have a potential implication on solvent-induced controlled molecular self-assembly. Water, which has the highest polarity, may lead to the formation of a stronger hydrogen binding network, which prompted transition of LM from individual nanospheres into fused spherical aggregates (FIG. 3A).

UV-vis absorption spectra of LM were also recorded in the different polar protic solvents, used for the self-assembly process. As the solvent's polarity increased, the absorption maximum of LM, corresponding to the intercomponent charge transfer transition, shifted towards longer wavelengths (327→347→364 nm) (FIG. 3B). These solvent-dependent spectral shifts arise from either non-specific interactions (dielectric enrichment) or specific bonds such as hydrogen bonding. This effect can be determined by using a solvent polarity scale or solvatochromic parameters. The excited states for most of the electronic transitions are more polar than their ground states because a greater charge separation is observed in the excited state. In a protic polar solvent the dipole-dipole interactions and hydrogen bonds stabilised the electronic excited state relative to the ground state; hence, the absorption in a more polar solvent will be shifted towards longer wavelengths (lower energy) in comparison with a less polar solvent.

To obtain an insight into the secondary structure of the different self-assembled structures of LM formed in the different solvents, Fourier transform infrared (FT-IR) analysis was generated and each spectrum was deconvoluted. The FT-IR spectra of the spherical structures formed in methanol exhibited two major peaks at 1633 cm⁻¹ and 1685 cm⁻¹, indicating an anti-parallel β-sheet structure (FIG. 3C). The spectra of the self-assembled structures (connecting spheres) formed in 50% ethanol exhibited one minor peak at 1615 cm⁻¹, which may relate either to extended hydrated structures or to a β-sheet and another major peak at 1667 cm⁻¹, which corresponds to β-turn configuration (FIG. 3D). The spherical aggregates formed in water exhibited one major peak at 1668 cm⁻¹ ascribed to β-turn and another minor peak at 1605 cm⁻¹, indicating a disordered and random structure (FIG. 3E).

The nature of the nanostructures formed by LM in water was further studied by X-ray powder diffraction (XRD) and compared to the assemblies in water formed by L. The XRD pattern indicated that both L and LM had amorphous and monoclinic crystalline structures. The XRD spectrum of LM exhibited sharp peaks along with the characteristic crystalline reflections in a wide range of 2θ (2°-40°); however, the intensities of these characteristic peaks were comparatively lower for L. Furthermore, the crystalline area and degree of crystallinity (%) were lower for L (200.2, 13.07%) compared with LM (305.6, 22.89%). In addition, the crystallite size for L and LM was 109.6 nm and 119.3 nm, respectively. Taken together, these structural analyses suggest that these spherical structures are not well ordered. However, it is noted that the spherical aggregates formed by LM in water are stable for at least one month at room temperature and atmospheric pressure.

### Optical Detection of DNA

Structures having aromatic moieties with π electron systems can interact with biomolecules such as DNA and proteins. In addition, some metal ions such as Zn²⁺ and Cu²⁺ are usually used as coordination centers, which have intrinsic fluorescence quenching properties. The detection or sensing of biological samples under physiological conditions (such as aqueous medium) is of major importance. Furthermore, the quenching efficiency of LM in water (spherical aggregates)>LM in ethanol (connecting spheres)>LM in methanol (individual spheres). This result suggests that the spherical aggregates formed by LM in water have a larger surface area for the adsorption of the probe DNA. This leads to a higher quenching efficiency with better optical response. Previous reports also showed that the association of ssDNA/GO is much faster than SWCNT and CNPs due to the presence of a larger adsorption area. It is therefore assumed that the surface area of the self-assembled nanostructures and the charge properties of the ssDNA are the main contributors to the adsorption rate. Hence, it is considered the metal-peptide complex, LM, with its self-assembled network as a smart sensing platform comparable to several MOFs.

The self-assembled LM was utilized as a biosensor, where Cu²⁺ acted as a coordination center, the π electron system mediated non-covalent bonding and fluorescein (FLC)-labeled ssDNA was used as a probe. The HIV-1U5 long terminal repeat sequence was used as the target analyte since early detection of HIV is highly important. Emission quenching was used as the basis for optical sensing.

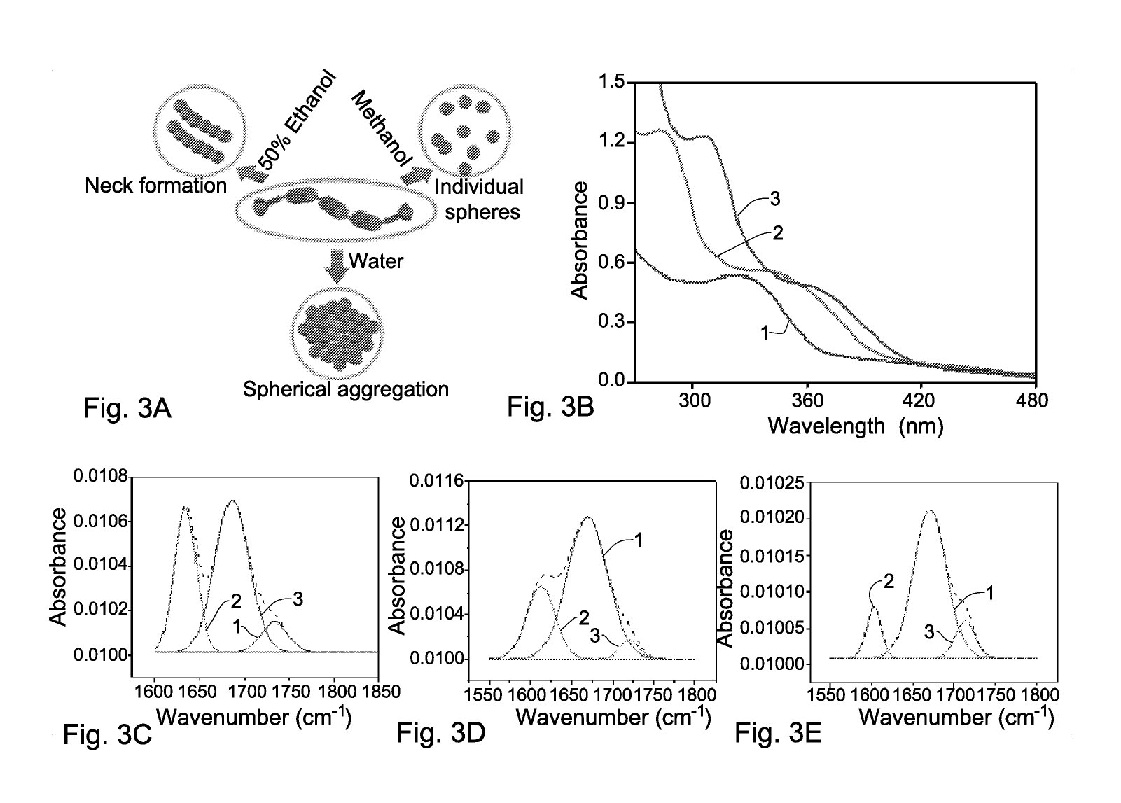

FIG. 4 provides a schematic representation of this optical detection platform. π-π interactions were assumed between the nucleobases of the DNA molecule and LM, along with the d⁹ metal (Cu²⁺) coordination of LM with the chelating site of the probe DNA, would bind the dye-labeled ssDNA and almost completely quench the fluorescence of the dye. The d⁹ metal center of Cu²⁺ can strongly quench the fluorescence of the dye owing to a photo-induced electron transfer process. The presence of a target molecule was assumed to alter the conformation of the dye-labeled ssDNA and that this conformational change will result in the release of the dye-labeled DNA in the duplex form, reversing the emission quenching effect. This result could give a turn-on emission response that is sensitive and selective to the target molecule. To validate our design, used following oligonucleotides were used: A Fluorescein (FLC)-labeled ssDNA sequence (P1) 5′-AGTCAGTGTGGAAAATCTCTAGC-FLC-3′, a synthetic oligonucleotide (T1) (5′-GCTAGAGATTTTCCACACTGACT-3′) from the HIV1-U5 long terminal repeat sequence as complementary target DNA, and the single-base mismatch sequence (M1) 5′-GCTAGAGATTGTCCACACTGACT-3′ (mismatch underlined). Four consecutive experiments were carried out to validate the feasibility and reproducibility of the experimental results.

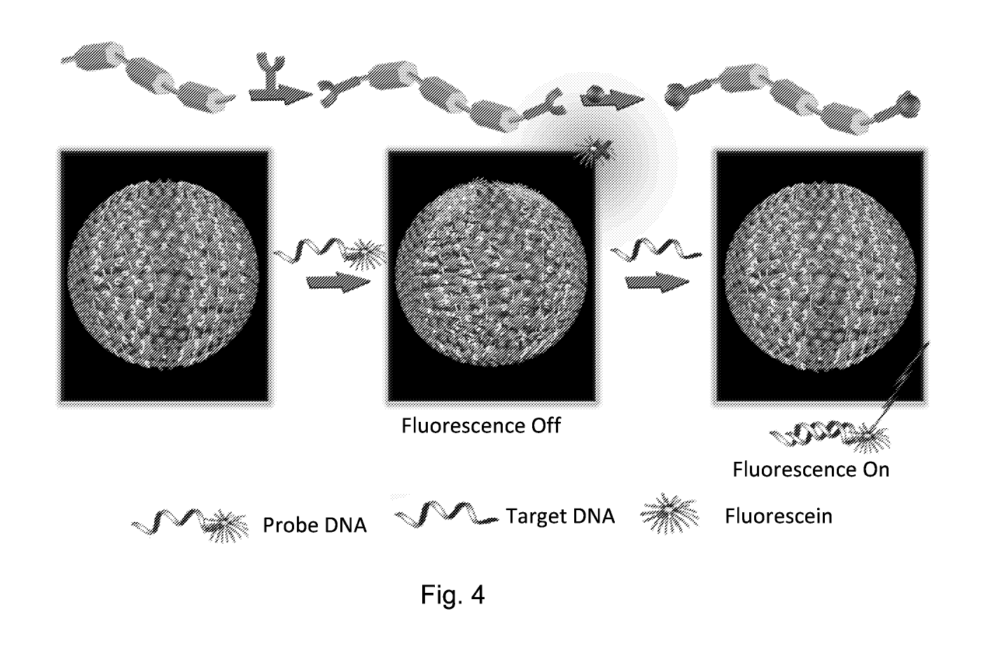

Since LM, the target and the probe DNA are water-soluble, the detection system is homogeneous. This type of homogeneous detection of nucleic acid with fluorescent probes has several natural advantages, such as ease of operation, rapid hybridization kinetics and potential compatibility with real-time monitoring and in situ cellular imaging. FIG. 5A shows the fluorescence emission spectra of P1 under different conditions. The probe DNA P1 (in Tris-HCl buffer pH=7.4) in the absence of LM had strong fluorescence emission owing to the presence of fluorescein (FIG. 5A, curve 1). In the presence of LM, up to 93% fluorescence quenching occurred (FIG. 5A, curve 4). This observation indicates a strong interaction between P1 and LM and high fluorescence quenching efficiency of LM. The P1-LM complex had a marked fluorescence enhancement upon the addition of the target DNA (T1, the complementary sequence of P1) (FIG. 5A, curve 3). The fluorescence of the free P1 was, however, barely influenced by the addition of only T1 (FIG. 5A, curve 2).

FIG. 5B illustrates the fluorescence intensity changes (F/F₀-1) for P1 and P1-LM upon the addition of different concentrations of T1, where F₀ and F are the fluorescence intensities at 521 nm in the absence and the presence of T1, respectively. With P1, no significant variation in the fluorescence intensity was found in the target concentration range. However, for the P1-LM conjugate, a dramatic increase in the fluorescence intensity was observed. In a reverse experiment, when we added LM to the mixture of P1/T1, the emission was barely affected and the quenching efficiency was significantly weaker than that in the presence of only P1. Thus, it may be concludes that LM weakly interacts with dsDNA. Due to this markedly weaker emission quenching, the ratio of the signal-to-background can be increased when LM is introduced. This result suggests that the sensing mechanism is based on the fluorescence quenching of the dye-labelled ssDNA (P1) upon binding to LM. More specifically, when P1 was hybridized with its complementary target T1 to form a duplex, it became rigid/stable, and conformational changes released the duplex from the LM network, reversing the emission quenching effect. Because the interactions between the double-stranded DNA (dsDNA) and LM were rather weak, it led to observable fluorescence that is the basis of a ‘signal-on’ DNA sensor.

The ssDNA adsorption on the GO surface is very fast and reaches equilibrium within one minute. The release of the duplex from the GO surface with a maximum emission recovery occurs within 30 minutes with a lower detection limit (LOD) of 10 nM. The emission quenching of the dye-labelled ssDNA by SWCNT exhibits a rapid reduction in the first hour followed by a slow decrease over the subsequent 2-3 hr. The release of the duplex from the wall of CNT in the presence of the complementary target reaches a plateau after 3 hr with an LOD of 4 nM. The CNPs reaches about 82% fluorescence quenching within 30 minutes and DNA hybridization occurs at a rate comparable to ssDNA adsorption and yields the best fluorescence response after 40 minutes (LOD>10 nM). Cu(H₂dtoa)-based MOF sensing of ssDNA has a quenching efficiency of 84.5% and the incubation time considered for maximum emission recovery is more than 4 hr with an LOD of 3 nM.

For better quantification of the target DNA, several factors were optimized, such as the incubation time and the dosage of LM. The effect of the dosage of LM was studied (FIG. 4C). The fluorescence intensity of 50 nmol probe DNA (P1) decreased with increasing LM dosage from 0-27 μL (the concentration of the LM stock solution was 1 mg mL⁻¹, 0.539 mM) and then reached a plateau. When 27 μL of LM was added, about 90% of the fluorescence emission quenching was observed. Hence, 27 μL (stock solution 0.539 mM) of LM was chosen as the standard dosage for the following experiments. The kinetic behaviour of P1 and LM as well as P1-LM conjugate in the presence of T1 was studied by monitoring the emission intensity as a function of time (FIG. 4D). The fluorescence of P1 was quenched completely by LM within 30 min; therefore, 30 min was chosen as the quenching time for subsequent experiments. The interaction of ssDNA with LM was rapid at room temperature and reached equilibrium within 30 minutes. However, the formation and release of dsDNA from LM was relatively slow. The dynamic behaviour of this sensing technique was studied by monitoring the fluorescence recovery of P1 as a function of incubation time in the presence of T1 (FIG. 4D). This analysis revealed that an increase in the incubation time results in a gradual increase in the fluorescence intensity and that it achieved a plateau after three hours. Hence, three hours was chosen as the optimum incubation time of the target DNA (T1).

FIG. 5A describes the fluorescence spectra of the FLC-labelled P1-LM complex conjugate upon the addition of increasing concentrations of the target DNA (T1). Increasing the concentration target DNA induced a gradual increase in the fluorescence intensity, implying that more P1 was released from the LM network. FIG. 5B shows that a linear relationship exists between the change in the fluorescence intensity (F_{T}-F_{M})/F_{M} and the concentration of target ss-DNA (T1) in the range of 4 nM to 100 nM, where F_{M} and F_{T} were the fluorescence intensities at 521 nm in the absence and presence of T1 calculated by:

(F_{T}−F_{M})/F_{M}=0.0022C+0.0607, R=0.993,

where C is the concentration of the target DNA and R is the regression coefficient of the equation. Based on this method, the detection limit of the target ss-DNA was estimated to be 1.4 nmol L⁻¹ (defined as S/N=3). This detection limit is lower than those obtained by systems that rely on carbon nanostructures including SWNT, GO, CNPs platform and electrochemical-DNA sensors. The major limit of carbon-based nanomaterials such as SWCNT, GO, and CNPs is their lack of (or low) solubility in water. Therefore, sensing platforms that are based on these materials cannot support homogeneous detection of biomolecules. Homogeneous detection technique has potential compatibility in detecting lower concentrations of targeted molecules with better sensitivity and in-situ cellular imaging. Moreover, this detection system, which we propose, exhibits excellent reproducibility because the relative standard deviation (RSD) for parallel experiments of four newly prepared ss-DNA solutions (100 nmol L⁻¹) was only 6.1%.

Usually, DNA-intercalating dyes can interact in a non-specific manner with DNA fragments and proteins. This greatly limits the application of the intercalating dyes in DNA detection. To evaluate the specificity of the system, an attempt was made to detect a one-base mismatched DNA fragment (MT1). In the presence of MT1, the fluorescent recovery was low. This indicates lower (F_{T}-F_{M})/F_{M} owing to the existence of an unstable duplex conformation (FIG. 5C). The introduction of the complementary DNA (T1) resulted in a marked fluorescence recovery and a larger (F_{T}-F_{M})/F_{M}. This result shows that a one-base mismatch in the target DNA sequence can be discriminated by the system. Furthermore, in order to confirm the specificity of this detection system, the emission spectra of P1 was recorded in the presence of MT1. The emission of the labelled dye was barely affected. However, the introduction of MT1 to the LM-P1 conjugate failed to produce a significant emission recovery. This is because MT1 cannot hybridize and release the probe ssDNA from LM by forming a stable duplex conformation or because LM possibly competitively destabilizes the mismatched duplex, which leads to insignificant emission recovery. This DNA-sequence signal specificity is higher than that of linear DNA probes, which cannot discriminate single-base mismatch targets. A further investigation was carried out in the presence of several possible interfering proteins such as bovine serum albumin (BSA), alkaline phosphatase (ALP), pepsin and lysosome (FIG. 5D). The interaction of ss-DNA with proteins mainly occurs through non-covalent binding; however, owing to the stability of the dsDNA structure formed between P1 and the perfectly complementary T1, its emission response (F/F₀) was not affected by the coexisting proteins, showing the good specificity of the present strategy.

Moreover, when L was used instead of LM in this sensing system, an insignificant emission response of P1 was observed. This experimental observation suggests that the low optical response of P1 in the presence of L is attributed to the absence of Cu(II) from the network. Cu(II) in LM has a d⁹ electronic configuration that facilitates its binding to the probe DNA (P1), along with π-π stacking, and it quenches the fluorescence of FLC owing to the photoinduced electron transfer (PET) process. It is well known that Cu(II) complexes with free coordination sites efficiently quench the emission of fluorophores with the binding properties in molecular beacon oligonucleotide probes; this triggers PET. The advantage of these types of optical quenchers is their ability to interact reversibly with probe DNA through space or weak contact interactions. This makes the detection of targeted DNA by an optical sensing stand feasible. The results from these studies suggest that LM can differentiate between ssDNA and ds DNA. This does not only offer selective detection of DNA—it may be possible to extend this optical sensing application to a wide spectrum of analytes by complementing LM with functional nucleic structures (e.g., aptamers). More importantly the emission quenching response of P1 after introducing LM in the presence of polyanions such as polyacrylic acid is comparable to that without polyacrylic acid. Further addition of increasing concentrations of T1 leads a steady increase in the emission recovery. This result suggests that this newly developed receptor sensing stand is stable and functions in the presence of polyanions such as polyacrylic acid.

### Experimental Section

### Materials

All chemicals and solvents are commercially available and were used as supplied unless otherwise stated Amino acids such as Phenylalanine and Glycine were purchased from Novabiochem (Darmstadt, Germany) Cu(ClO4)₂ was purchased from Sigma Aldrich (St Louis, Mo., USA). 2-(chloromethyl) pyridine hydrochloride was purchased from Sigma Aldrich (St Louis, Mo., USA), aniline, ethylenediamine and phosphoryl chloride were purchased from Merck (Darmstadt, Germany) (1-Hexadecyl) trimethyl ammonium chloride was purchased from Alfa Aser (Ward Hill, China), N,N′-dicyclohexyl-carbodiimide, Di-tert-butyl dicaronate and 1,1,1,3,3,3-hexafluoro-2-propanol (HFP) were purchased from Alfa Aser (Heysham, England); hydroxybenzotriazole was purchased from Chem-Implex International (Wood Dale, Ill., USA). The following DNA sequences were synthesized by Sigma—(Rehovot, Israel):

Probe DNA: 5-AGTCAGTGTGGAAAATCTCTAGC-FLC-3′ (FLC=Fluorescein) Target DNA: 5′-GCTAGAGATTTTCCACACTGACT-3′ Single-base mismatch DNA: 5′-GCTAGAGATTGTCCACACTGACT-3′. The probe DNA (P1) is the complementary sequence of HIV-1 U5. The target DNA (T1) is the HIV-1 U5 sequence. Single-base mismatched DNA has a single-base mismatch in the underlined position.

### Self-Assembly of L and LM

A fresh stock solution of L and LM was prepared by dissolving the lyophilised forms of L and LM in HFP to a concentration of 100 mg/mL. Then, we blended these peptides in several different proportions and diluted them in the desired solvent as indicated in Table 1. The polarized solvent allowed the molecules to self-assemble.

### Preparation of the Complex Probe DNA-LM and Assay of the Target DNA

First, 1.0 mg of LM (which was previously self-assembled in water and dried) was dispersed in 1 mL triple distilled water (TDW) by sonication. Next, 27 μL of this suspension were mixed with 50 nM probe DNA with oscillation at RT in order to obtain a symmetrical and limpid solution. The fluorescence intensity of the solution was detected; then different concentrations of the target DNA were added to the solution containing P1-LM complex with oscillation at 35° C. for 3 h (except for the time-course study). The complex obtained was used immediately to measure fluorescence. DNA fragments in buffer solutions were prepared by dissolving the DNA in 0.5 mM Tris-HCl buffer (pH 7.4, containing 100 mM NaCl and 5 mM MgCl₂).

### High-Resolution Scanning Electron Microscopy (HR-SEM)

A 10 μL drop of the solution of either L or LM in the different solvents was placed on a glass cover slip and allowed to dry at RT. The substrates were then coated with gold using a Polaron SC7640 Sputter Coater. SEM analysis was performed using a high-resolution scanning electron microscope (HR-SEM, Serion equipped with X-MAX20 SDD Inca 450 EDS LN² free detector) operating at 1 kV.

### Transmission Electron Microscopy (TEM)

A 10 μL drop of the solution of either L or LM in the different solvents was placed on a 200-mesh copper grid, covered by carbon-stabilized Formvar® film (Electron Microscopy Science, PA, USA). After 1 min, excess fluid was removed from the grid. The samples were analysed using a Tecnai T12 G² Spirit (Cryo-TEM) operating at 120 kV.

### Atomic Force Microscopy Analysis

Topography images of the structures on glass cover slips were taken using JPK NanoWizard®3 (JPK instruments, Germany) working in AC mode. Si₃N₄ cantilever probes with a spring constant of 3 Nm⁻¹ and a resonance frequency of 75 kHz were used.

### Fourier Transform Infrared Spectroscopy (FT-IR)

Fourier transform infrared spectra were recorded using a Nicolet 6700 FT-IR spectrometer with a deuterated triglycine sulfate (DTGS) detector (Thermo Fisher Scientific, Mass., USA). The metal-peptide complex solutions were deposited on a CaF₂ window and dried under vacuum. The peptide deposits were resuspended with D₂O and subsequently dried to form thin films. The re-suspension procedure was repeated twice to ensure a maximal hydrogen-to-deuterium exchange. The measurements were taken using 4 cm⁻¹ resolution and averaging 2000 scans. The transmittance minimal values were determined by the OMNIC analysis program (Nicolet).

### UV-Vis Spectroscopy

UV-Vis absorption spectra of the monomeric form of L, LM in 1,1,1,3,3,3-hexafluoro-2-propanol (HFP) and the UV-Vis absorption spectra of self-assembled metallo peptides (LM) in the different solvents (methanol, 50% ethanol and water) were recorded using a UV/Vis spectrophotometer (SHIMADZU, UV-1650PC).

### Fluorescence Spectroscopy

For the quantitative homogeneous detection of the target DNA, the fluorescence measurements were performed at RT using a fluorescent spectrometer (Perkin Elmer LS 55). The emission spectra were collected from 500 to 650 nm with an excitation wavelength of 494 nm. Both the excitation and emission slit widths were set to 5.0 nm. The fluorescence intensity at 521 nm was used for quantitative analysis. The quenching efficiency (Q_{E},%) was calculated by the formula: Q_{E}=(1−F_{M}/F₀)×100%, where F_{M} and F₀ are fluorescence intensities at 521 nm in the presence and the absence of LM. The fluorescence recovery was calculated by the formula: RE=(F_{T}/F_{M}−1)×100%, where F_{T} and F_{M} are fluorescence intensities at 521 nm in the presence and the absence of the target DNA after introducing LM.

### X-Ray Diffraction (XRD) Analysis

The phase of the product was identified by X-ray powder diffraction (X-Ray Diffractometer—D8 Advance), using Cu K α (λ=0.15406 nm) and a solid state NaI dynamic scintillation detector. The full Diffrac^{Plus} package software was used for data acquisition, phase analysis, crystallography and thin film characterisation.

### Dynamic Light Scattering (DLS) Analysis

Dynamic light scattering measurements of the assemblies were performed using a Nano-zeta sizer (Malvern instruments), model ZEN3600).

### Peptides Synthesis:

Peptides were synthesized by conventional solution-phase methods. Peptide coupling was mediated by dicyclohexylcarbodiimide/1-hydroxybenzotriazole (DCC/HOBt). The products were purified by column chromatography using silica gel (100-200 mesh) as the stationary phase and an n-hexane-ethyl acetate mixture as an eluent. The final compounds were fully characterized by Bruker 500 MHz 1H-NMR spectroscopy, and mass spectroscopy (Applied Biosystems Voyager-DE Pro MALDI-TOF and Accela Autosampler, Thermo Scientific (CCQ Fleet)).

Synthesis of BOC Phe-OH: A solution of L-phenylalanine (3.30 g, 20 mmol) in a mixture of dioxane (40 mL), water (20 mL) and 1 M NaOH (20 mL) was stirred and cooled in an ice-water bath. Di-tertbutylpyrocarbonate (4.583 g, 21 mmol) was added and stirring continued at room temperature (RT) for 6 h. Then the solution was concentrated in vacuum to about 10-15 mL, cooled in an ice-water bath, covered with a layer of ethyl acetate (about 50 mL) and acidified with a dilute solution of KHSO4 to pH 2-3 (determined by Congo red). The aqueous phase was extracted with ethyl acetate and this was done repeatedly. The ethyl acetate extracts were pooled, washed with water, dried over anhydrous Na₂SO₄ and evaporated in a vacuum. The pure material was obtained as a waxy solid. Yield 4.45 g (16.8 mmol, 84.0%).

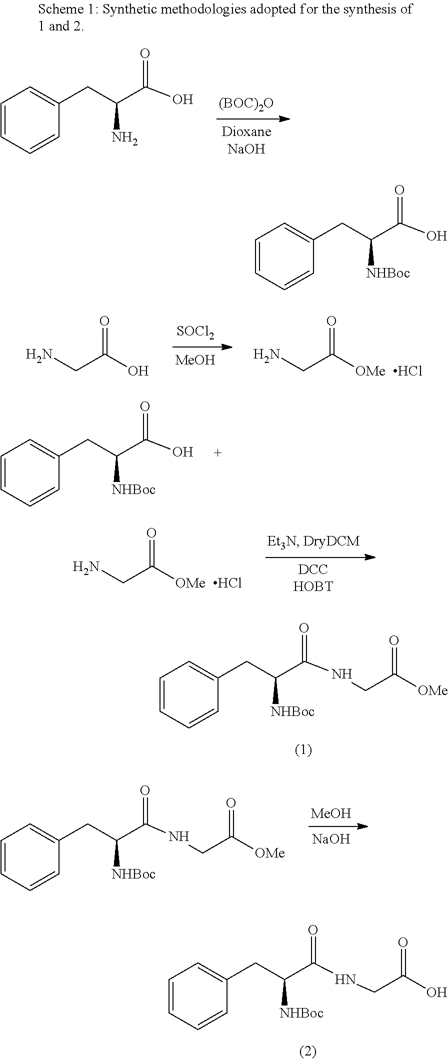

Synthesis of NH₂-Gly-OMe Hydrochloride: 4.5 g (60 mmol) of L-glycine was dissolved in 90 mL of MeOH and cooled in an ice bath. Then, 12 ml of SOCl2 was added dropwise and stirred for 8 h. The excess solvent was evaporated under rotary vacuum. The dried crystalline solid product obtained was L-glycine methyl ester hydrochloride. Yield 6.30 g (50.4 mmol, 85.0%).

Synthesis of BOC-Phe-Gly-OMe (1): 4.0 g (15 mmol) of Boc-Phe-OH was dissolved in 40 ml dry DCM in an ice-water bath. H-gly-OMe. HCl 2.507 g (20.0 mmol) and Et₃N 4 ml, 30 mmol) were then added to the reaction mixture, followed immediately by the addition of 3.30 g (16.0 mmol) dicyclohexylcarbodiimide (DCC) and 2.16 g (16.0 mmol) of HOBt. The reaction mixture was allowed to warm-up to RT and was stirred for 48 h. DCM was evaporated and the residue was dissolved in ethyl acetate (60 mL). The dicyclohexylurea (DCU) was filtered off. The organic layer was washed with 2M HCl (3×50 mL), brine (2×50 mL), 1 M sodium carbonate (3×50 mL) and brine (2×50 mL) and finally dried over anhydrous sodium sulfate. It was then evaporated under vacuum to yield Boc-Phe-Gly-OMe as a white solid. The product was purified by silica gel (100-200 mesh) using n-hexane-ethyl acetate (3:1) as eluent. Yield: 3.65 g (10.85 mmol, 72.35%). ¹H NMR (CDCl₃, 400 MHz, δppm): 7.31-7.28 (m, 2H, ArH of Phe), 7.23-7.20 (m, 3H, ArH of Phe), 6.52 (b, 1H, NH Phe), 5.05 (b, 1H, NH Gly), 4.42-4.41 (m, 1H, CαH, Phe) 4.07-3.91 (dd, 2H, —CH2-Gly), 3.73 (s, 3H, OMe), 3.14-3.05 (m, 2H, CβH, Phe) 1.39 (s, 9H, Boc). ESI-MS (m/z): [M]=336.38 (calculated); 336.59 (observed), [M+Na+H]+=360.38 (calculated); 360.11 (observed); [M+K+H]+=376.38 (calculated); 376.29 (observed), [M+2Na]+=382. 38 (calculated); 381.21 (observed).

Synthesis of BOC-Phe-Gly-OH (2): To 3.0 g (8.91 mmol) of Boc-Phe-Gly-OMe, 30 mL MeOH and 2M 15 mL NaOH were added and the progress of saponification was monitored by thin layer chromatography (TLC). The reaction mixture was stirred. After 10 h, the methanol was removed under vacuum; the residue was dissolved in 50 mL of water and washed with diethyl ether (2×50 mL). Then, the pH of the aqueous layer was adjusted to 2 using 1M HCl and extracted with ethyl acetate (3×50 mL). The extracts were pooled, dried over anhydrous sodium sulfate and evaporated under vacuum to obtain the compound as a waxy solid. Yield: 2.72 g (8.46 mmol, 95%). ¹H NMR (DMSO-d₆, 400 MHz, δppm): 12.56 (s, 1H, COOH), 8.23 (t, 1H, NH Gly), 7.27, 7.16 (m, 5H, ArH of Phe), 6.89 (d, 1H, NH Phe), 4.23-4.17 (m, 1H, CαH, Phe) 3.86-3.72 (m, 2H, —CH2— Gly), 3.03-2.69 (m, CβH, Phe) 1.28 (s, 9H, Boc). ESI-MS (m/z): [M+Na+H]+=346.35 (calculated); 346.42 (observed); [M+K+H]+=362.35 (calculated); 362.37 (observed).

Synthesis of BOC-Phe-NH—CH₂—CH₂—NH-Phe-Boc (3) and NH2-Phe-NH—CH₂—CH₂—NH-Phe-NH₂ (4): 3.0 g (11.27 mmol) of Boc-Phe-OH were dissolved in 40 ml dry DCM in an ice-water bath. Ethylenediamine (340 mg) (5.63 mmol) was then added to the reaction mixture, followed immediately by the addition of 2.79 g (13.5 mmol) dicyclohexylcarbodiimide (DCC) and 1.82 g (13.5 mmol) of HOBt. The reaction mixture was allowed to warm-up to RT and was stirred for 48 h. DCM was evaporated and the residue was dissolved in ethyl acetate (60 mL) and the dicyclohexylurea (DCU) was filtered off. The organic layer was washed with 2M HCl (3×50 mL), brine (2×50 mL), 1 M sodium carbonate (3×50 mL) brine (2×50 mL) and dried over anhydrous sodium sulfate; finally it was evaporated under vacuum to yield BOC-Phe-NH—CH2—CH2—NH-Phe-Boc (3) as a white solid. The product was purified by silica gel (100-200 mesh) using n-hexane-ethyl acetate (4:1) as eluent. Yield: 2.04 g (3.61 mmol, 64.18%). ¹H NMR (CDCl₁₃, 400 MHz, δppm): 7.34-7.30 (m, 3H, ArH of Phe), 7.19-7.17 (m, 2H, ArH of Phe), 5.77 (b, 1H, NH Phe), 5.10 (b, 1H, NH Ethelynediamine), 4.77-4.11 (m, 1H, CαH, Phe) 3.18-3.16 (m, 2H, —CH₂-Ethylenediamine), 3.02-2.93 (m, 2H, CβH Phe) 1.41 (s, 9H, Boc). ESI-MS (m/z): [M+Na+2H]+=579.67 (calculated); 579.74 (observed); [M+K+H]+=595.67 (calculated); 596.10 (observed).

Next, 2 g (3.60 mmol) of compound 3 were dissolved in 25 mL of DCM in an ice bath. Then, 6 mL of TFA were added and stirred for 2h. The progress of the reaction was monitored by TLC. After the reaction was completed, all solvents were evaporated in a rotary evaporator. The product was then dissolved in water, neutralized with NaHCO₃ solution, extracted with ethyl acetate, dried over anhydrous sodium sulphate and evaporated by rotary evaporator to obtain an oily product 4, which was immediately used for the next reaction. Yield: 1.194 g (3.36 mmol, 93.6%). ESI-MS (m/z): [M+Na+2H]+=379.44 (calculated); 379.93 (observed); [M+2Na]+=400.44 (calculated); 400.25 (observed).

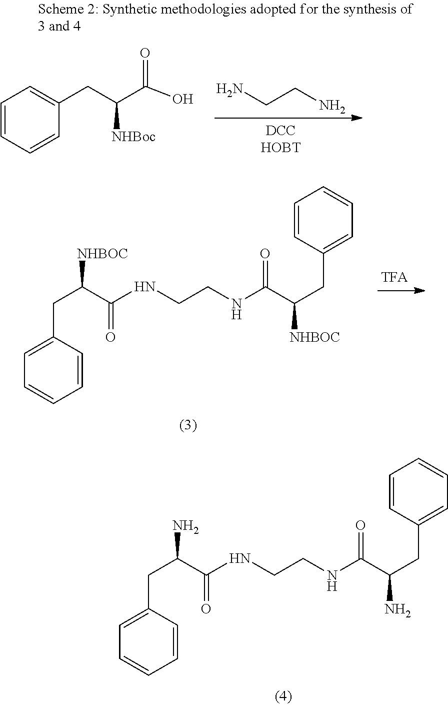

Synthesis of BOC-Phe-Gly-Phe-NH—CH₂—CH₂—NH-Phe-Gly-Phe-Boc (5) and NH₂-Phe-Gly-Phe-NH—CH₂—CH₂—NH-Phe-Gly-Phe-NH2 (6): 2.554 g (7.90 mmol) of Boc-Phe-Gly-OH were dissolved in 30 ml dry DCM in an ice-water bath. Compound 4 (1.274 g 3.6 mmol) was then added to the reaction mixture, followed immediately by the addition of 1.96 g (9.48 mmol) dicyclohexylcarbodiimide (DCC) and 1.28 g (9.48 mmol) of HOBt. The reaction mixture was allowed to warm-up to RT and stirred for 48 h. DCM was evaporated and the residue was dissolved in ethyl acetate (50 mL) and the dicyclohexylurea (DCU) was filtered off. The organic layer was washed with 2M HCl (3×50 mL), brine (2×50 mL), 1 M sodium carbonate (3×50 mL), brine (2×50 mL), dried over anhydrous sodium sulfate and evaporated in a vacuum to yield BOC-Phe-Gly-Phe-NH—CH₂—CH₂—NH-Phe-Gly-Phe-Boc (5) as a white solid. The product was purified by silica gel (100-200 mesh) using n-hexane-ethyl acetate (4:1) as eluent. Yield: 2.09 g (2.20 mmol, 62.21%). ¹H NMR (DMSO-d₆, 400 MHz, δppm): 8.11-8.05 (m, 2H, NH Phe and Gly), 7.28-7.23 (m, 8H, ArH Phe), 7.22-7.18 (m, 4H, ArH Phe), 6.97 (d, J=8.3 Hz, 1H, NH Phe), 4.46-4.42 (m, 1H, CαH, Phe) 4.22-4.17 (m, 1H, CαH Phe), 3.83-3.78 (dd, 1H, CH2 Gly), 3.69-3.63 (dd, 1H, CH2 Gly) 3.09-2.96 (m, 4H, CβH Phe), 2.87-2.36 (m, 2H, CH₂ EDA), 1.30 (s, 9H, Boc). ESI-MS (m/z): [M+2H]+=965.13 (calculated); 965.61 (observed); [M+H3O]+=982.13 (calculated); 982.60 (observed); [M+Na+H]+=987.14 (calculated); 987.44 (observed).

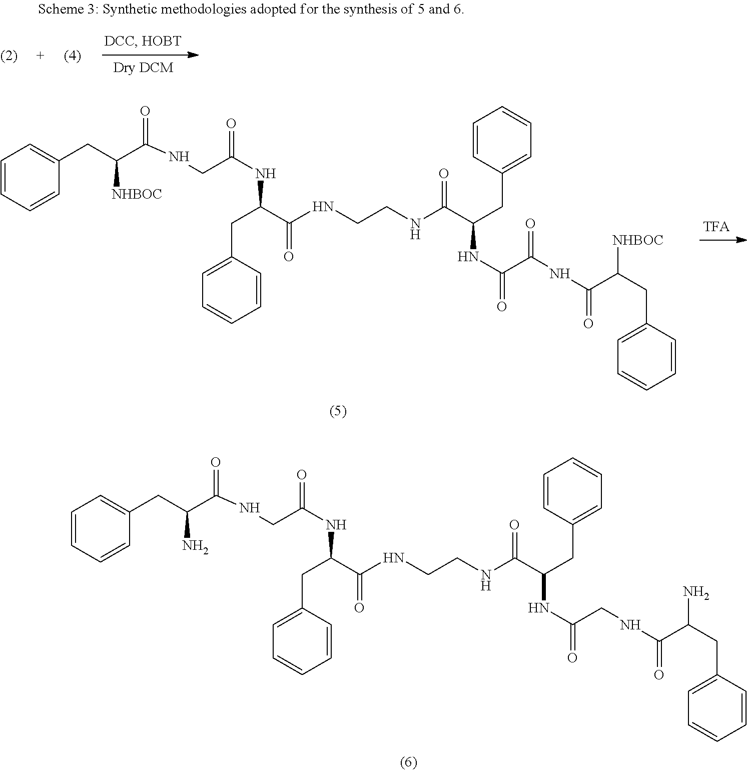

Next, 1.5 g (1.58 mmol) of compound 5 was dissolved in 20 mL of DCM in an ice bath. Then, 4 mL of TFA were added and stirred for 2 h. The progress of the reaction was monitored by TLC. After the reaction was completed, all solvents were evaporated in a rotary evaporator. The product was then dissolved in water, neutralised with NaHCO3 solution, extracted with ethyl acetate, dried over anhydrous sodium sulphate, and evaporated by a rotary evaporator to obtain an oily product 6, which was immediately used for the next reaction. Yield: 1.103 g (1.47 mmol, 93.3%). ESI-MS (m/z): [M]=762.89 (calculated); 762.75 (observed); [M+Na]+=785.89 (calculated); 785.80 (observed); [M+K]+=801.89 (calculated); 801.62 (observed).

Synthesis of Phenyl-bis-pyridin-2ylmethyl-amine (1/): To a solution of 2-chloromethylprydine hydrochloride (2 g, 12 mmol) in H₂O (0.5 ml), aniline (0.558 g, 6 mmol), 5 N NaOH (6 ml) and hexadecytrimethylammonium chloride (20 mg) were added under N₂ protection. The mixture was stirred vigorously for 24 h at RT. It was then extracted with CH₂Cl₂, and the extract was washed with H₂O and dried with MgSO₄. After the solvent was evaporated, the desired product was obtained as a beige solid via column chromatography (silica, CH₂Cl₂/AcOEt, 4/1, v/v). Yield: 850.6 g (3.08 mmol, 51.2%). ¹H NMR (CDCl₃, 400 MHz, δppm): 8.60 (d, J=6.8 Hz, 2H ArH), 7.63 (t, J=7.6 Hz, 2H ArH), 7.28 (d, J=8 Hz, 2H ArH), 7.19-7.15 (m, 4H ArH), 6.74-6.70 (m, 3H ArH), 4.84 (s, 4H —CH2—). ESI-MS (m/z): [M+H]+=276.14 (calculated); 276.17 (observed).

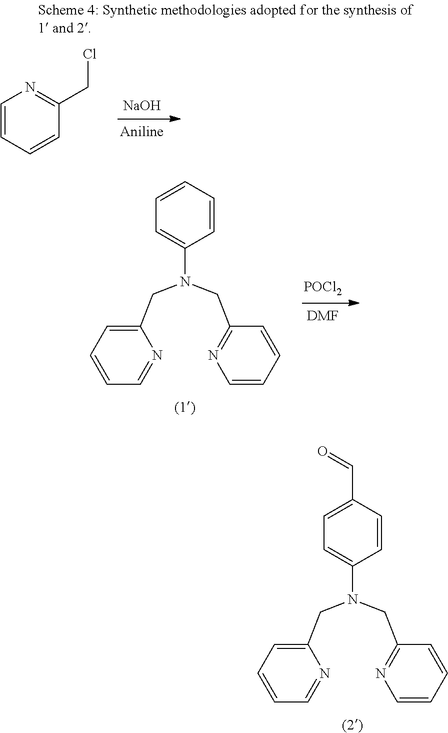

Synthesis of 4-(Bis-Pyridin-2-ylmethyl-amino-benzaldehyde (2′): POCl₃ (1 ml, 17 mmol) was added to the solution of DMF (2 ml, 26 mmol) in 2 portions within 30 min, and cooled in an ice bath. Then, the solution was stirred for 30 min. Compound 1′ (0.800 g, 2.89 mmol) in DMF (1.25 ml) was added in portions within 20 min. The mixture was heated for 3 h at 90° C., poured into H₂O (5 ml), and then neutralized to pH 6-8 with K₂CO₃ along with stirring. The mixture was extracted with CH2Cl2, and dried with Na₂SO₄. Via column chromatography (silica, petroleum:acetone, 5:3, v/v), the desired product was obtained as a yellow sticky oil. Yield: 294.3 mg (0.97 mmol, 33.5%). ¹H NMR (CDCl₃, 400 MHz, δppm): 9.75 (s, 1H, —CHO), 8.60 (d, J=5.8 Hz, 2H ArH), 7.68-7.61 (m, 4H ArH), 7.20 (d, J=7.8 Hz, 4H ArH), 6.78 (d, J=8.4 Hz, 2H ArH), 4.89 (s, 4H —CH2—). ESI-MS (m/z): [M]=303.13 (calculated); 303.33 (observed).

Synthesis of L: 2′ (200 mg, 0.65 mmol) was added to a solution of 6 (490.2 mg, 0.655 mmol) dissolved in 20 ml of methanol. The resulting mixture was stirred for 8 h. After the reaction was completed (confirmed by TLC), the reaction mixture was filtered off and a light brown oily residue was obtained upon removal of the solvent from the filtrate under vacuum. This oily residue upon treatment with n-hexane yielded a light brownish solid precipitate, which was collected by decantation as well as proper washing and drying to afford L as a pure product. Yield: 518.6 mg, 60.5%. 9.04 (d, J=6.0 Hz, 2H ArH of DPA based receptor), 8.59 (s, 1H N═CH), 8.13-8.11 (m, 2H, NH Phe and Gly), 7.71-7.66 (m, 14H ArH), 7.22 (d, J=7.8 Hz, 4H ArH), 6.73 (d, J=8.2 Hz, 2H ArH), 5.32 (s, 4H, —CH2— of DPA), 4.12-4.07 (m, 1H, CαH Phe), 3.89-3.84 (m, 1H, CαH Phe), 3.54-3.16 (m, 2H, CH₂ Gly), 2.38-2.35 (m, 4H, CH Phe), 2.12-2.09 ((m, 2H, CH2 EDA), ESI-MS (m/z): [M/2]=666.32 (calculated); 666.37 (observed); [M+2H]+=1335.58 (calculated); 1335.64 (observed).

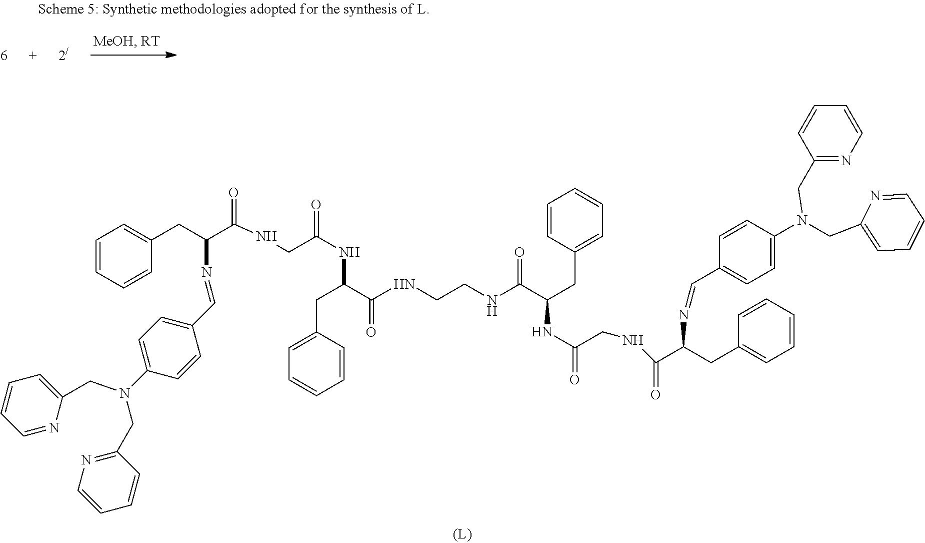

Synthesis of LM: L (200 mg, 0.15 mmol) was dissolved in 20 mL of methanol and then a solution of Cu(ClO₄)₂.6H₂O (138 mg, 0.372 mmol) in 5 mL HPLC water was added in a dropwise manner into it. The resulting solution was stirred for 10 h at RT. The desired compound was then precipitated in a pure form by slow evaporation of the solvent at RT. The precipitate was filtered, washed with cold water and dried. Yield: 160 mg, 57.5%. ESI-MS (m/z): [M+2H]+=1854.86 (calculated); 1855.31 (observed).

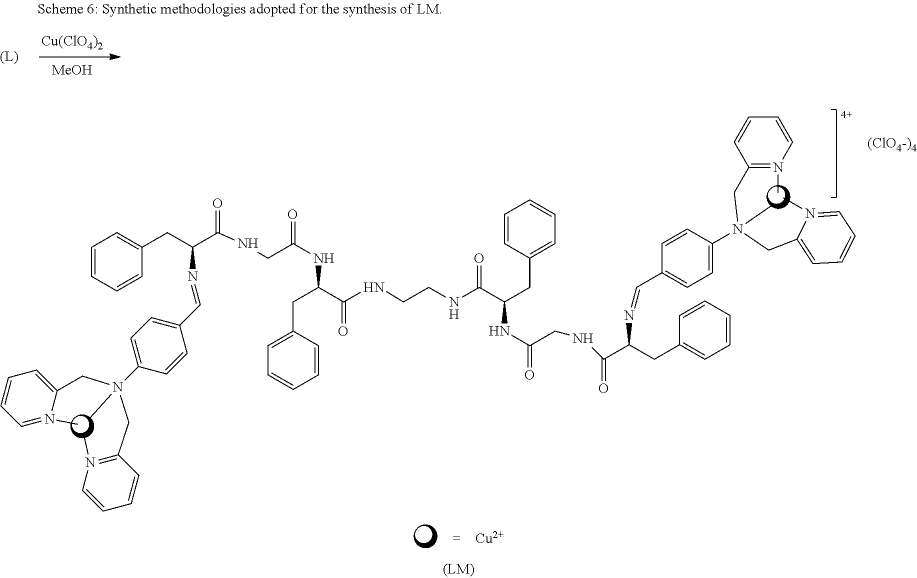

1-43. (canceled)

44. A biosensor comprising:
a peptide moiety; and
a moiety capable of altering fluorescence emission, the moiety comprising at least one metal ion,
the biosensor being associated with at least one probe molecule having a fluorescent label, the probe molecule being selected to partially or fully interact with at least one target molecule.

45. The biosensor according to claim 44, being in a form selected from tubular structure; fibrilar structures; spheres; joint spherical structures; spherical aggregates; distorted spherical aggregates; linear, two-dimensional or three-dimensional arrays; and single molecule forms.

46. The biosensor according to claim 44, self-assembled into a three dimensional form in solution.

47. The biosensor according to claim 44, wherein the peptide comprises an amino acid is selected amongst alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine valine, pyrrolysine and selnocysteine, or an amino acid analog selected from homo-amino acids, N-alkyl amino acids, dehydroamino acids, aromatic amino acids and α,α-disubstituted amino acids.

48. The biosensor according to claim 44, wherein the peptide comprises an aromatic amino acid.

49. The biosensor according to claim 44, wherein the peptide moiety comprises an amino acid selected from phenylalanine (Phe) and glycine (Gly).

50. The biosensor according to claim 49, wherein the peptide moiety comprises the amino acid motif Phe-Gly or Gly-Phe.

51. The biosensor according to claim 44, wherein the moiety capable of altering fluorescence emission is selected to modulate fluorescence emission from the florescence labeled probe molecule.

52. The biosensor according to claim 51, wherein modulation is selected from attenuation, quenching, enhancement, shift in wavelength, shift in polarity and changing fluorescence lifetime, or
wherein modulation is by quenching, and wherein the moiety capable of altering fluorescence emission is a fluorescence quenching moiety.

53. The biosensor according to claim 52, wherein the fluorescence quenching moiety is selected from aliphatic amines, aromatic amines, halogenated moieties, and electron scavengers.

54. The biosensor according to claim 44, wherein the at least one metal ion is an electron scavenger.

55. The biosensor according to claim 54, wherein the metal is Cu or Cd or Pd or Mn or Eu or As or Cs or Zn or Hg or Ni or Co.

56. The biosensor according to claim 54, wherein the at least one metal ion is directly associated with an amino acid of the peptide moiety or with a ligand group associated with an amino acid of the peptide moiety.

57. The biosensor according to claim 44, wherein one or both of said probe molecule and target molecule is a nucleic acid.

58. The biosensor accordion to claim 57, wherein the probe molecule is a probe nucleic acid and the target molecule is a target nucleic acid.

59. The biosensor according to claim 58, wherein the probe nucleic acid is selected to hybridize to the target nucleic acid through a portion or portions of the probe sequence that are substantially complementary to a target nucleic acid sequence.

60. The biosensor according to claim 51, wherein the labelled probe molecule is at least one nucleic acid associated with a fluorescence label.

61. The biosensor according to claim 57, wherein the probe nucleic acid and the target nucleic acid are each, independently, selected from DNA, single stranded DNA (ssDNA), double-stranded DNA (dsDNA), cDNA; and RNA.

62. The biosensor according to claim 61, wherein the probe nucleic acid and the target nucleic acid are each, independently, selected from aptamers, ribonucleotides, deoxyribonucleotides, ribonucleotide polyphosphate molecules, deoxyribonucleotide polyphosphate molecules, peptide nucleotides, nucleoside polyphosphate molecules, metallonucleosides, phosphonate nucleosides and modified phosphate-sugar backbone nucleotides.

63. The biosensor according to claim 58, wherein the probe nucleic acid is dsDNA and the target nucleic acid is a ssDNA.

64. A method of determining the presence of at least one target nucleic acid in a sample, the method comprising:
contacting said sample with at least one biosensor according to claim 44,
permitting association between a probe molecule and the target nucleic acid in the sample; and
measuring emission from the sample to determine the presence or absence of the at least one target nucleic acid in the sample;
whereby an emission of fluoresce indicates the presence of at least one nucleic acid in the sample.
